(19) United States
(12) Patent Application Publication
(10) Pub. No.: US 2024/0123046 A1
(43) Pub. Date: Apr. 18, 2024

(54) IMMUNOGENIC COMPOSITIONS

(71) Applicants: THE SCRIPPS RESEARCH INSTITUTE, La Jolla, CA (US); FRED HUTCHINSON CANCER RESEARCH CENTER, North Seattle, WA (US)

(72) Inventors: William R. SCHIEF, La Jolla, CA (US); Kristen COHEN, North Seattle, WA (US); Julie MCELRATH, North Seattle, WA (US)

(21) Appl. No.: 18/257,814

(22) PCT Filed: Dec. 17, 2021
(86) PCT No.: PCT/US2021/064110

§ 371 Date: Jun. 15, 2023

Related U.S. Application Data
(60) Provisional application No. 63/127,975, filed on Dec. 18, 2020.

(51) Int. Cl. A61K 39/02; C07K 14/005; C07K 14/74; C12N 9/10

(52) U.S. Cl. CPC A61K 39/0208; C07K 14/005; C07K 14/70539; C12N 9/1085; A61K 2039/53; C12Y 205/01078

(57) ABSTRACT

Provided herein are compositions comprising novel MHC class II T cell epitopes and method using thereof. In some embodiments, a composition described herein comprises an immunogenic polypeptide comprising an MHC class II T cell epitope and a target antigen. In some embodiments, the target antigen is a viral, bacterial, parasite or tumor specific antigen. In some embodiments, the target antigen comprises a virus, bacteria, parasite or tumor specific polypeptide. In some embodiments, the composition comprises a fusion polypeptide comprising the immunogenic polypeptide and the target polypeptide. Also provided are polynucleotides encoding the fusion polypeptide, and methods of administering a composition comprising the polynucleotide to a subject to elicit an immune response. In some embodiments, the polynucleotide is an RNA comprising modified ribonucleotides.

### CROSS-REFERENCE TO RELATED APPLICATIONS

This application claims the benefit of U.S. Application No. 63/127,975, filed Dec. 18, 2020, which is incorporated herein by reference in its entirety.

### GOVERNMENT INTEREST

This invention was made with government support under grant number AI144462 awarded by the National Institutes of Health. The government has certain rights in the invention.

### FIELD OF THE INVENTION

The field of the invention generally relates to MHC class II helper epitopes, immunogenic compositions comprising thereof, and methods of using the immunogenic compositions in eliciting an immune response.

### BACKGROUND

CD4+ helper T cells play a critical role in the maturation of antibody responses. Helper T cells are first primed by dendritic cells and other APCs and then further activated by germinal center B cells. In the germinal center, interaction between CD4+ helper T cells and germinal center B cells enhance survival, differentiation, somatic hypermutation, and class switching in the B cells. Provision of T cell help, however, is contingent upon CD4+ helper T activation by germinal center B through T cell receptor (TCR) peptide-MHC class II interaction. As such, robust germinal center B cell responses are dependent on presentation of MHC class II-restricted epitope, derived from the antigen, by germinal center B cells to helper T cells. However, different epitopes have varying affinity for binding to MHC class II receptors depending on the hosts' haplotype such that peptide vaccines as well as smaller protein domains may not intrinsically contain a potent CD4+ helper epitope to drive germinal center responses.

Incorporation and fusion of a potent CD4+ helper epitope with the target antigen can provide a simple and effective strategy to enhance the induced humoral immunity. Several important epitopes have been identified in this manner. For example, the Pan DR epitope (PADRE) has improved immunogenicity of peptide and protein vaccines in animal studies.

There is a need in the art for the identification of new MHC class II CD4-helper epitopes that can be used to increase the immune responses induced by various antigens.

### BRIEF SUMMARY

In one aspect, provided herein are immunogenic polypeptides comprising an MHC class II T cell epitope, wherein the peptide comprises an amino acid sequence of a fragment of A. aeolicus lumazine synthase.

In one aspect, provided herein are immunogenic polypeptides comprising one or more MHC class II T cell epitope, wherein the MHC class II T cell epitope comprises an amino acid sequence selected from the group consisting of

(SEQ ID NO: 1)
a) ATPHFDYIASEVSKG
(LumSyn22) comprising 0, 1, 2, 3, 4, or 5
substitutions,
(SEQ ID NO: 2)
b) FGVITADTLEQAIER
(LumSyn29) comprising 0, 1, 2, 3, 4, or 5
substitutions,
(SEQ ID NO: 3)
c) FDYIASEVSKGLADL
(LumSyn23) comprising 0, 1, 2, 3, 4, or 5
substitutions,
(SEQ ID NO: 4)
d) ATPHFDYIASEVSKGLADL
(LumSyn22/23) comprising 0, 1, 2, 3, 4,
or 5 substitutions,
e) 9, 10, 11, 12, 13, 14, or 15 consecutive
residues of
(SEQ ID NO: 1)
ATPHFDYIASEVSKG
(LumSyn22),
f) 9, 10, 11, 12, 13, 14, or 15 consecutive
residues of
(SEQ ID NO: 2)
FGVITADTLEQAIER
(LumSyn29),
g) 9, 10, 11, 12, 13, 14, or 15 consecutive
residues of
(SEQ ID NO: 3)
FDYIASEVSKGLADL
(LumSyn23),
and
h) 10, 11, 12, 13, 14, 15, 16, 17, 18, or
19 consecutive residues of
(SEQ ID NO: 4)
ATPHFDYIASEVSKGLADL
(LumSyn22/23).

In some embodiments, the MHC class II T cell epitope comprises an amino acid sequence selected from the group consisting of a) ATPHFDYIASEVSKG (SEQ ID NO:1), b) FGVITADTLEQAIER (SEQ ID NO:2), c) FDYIASEVSKGLADL (SEQ ID NO:3), and d) ATPHFDYIASEVSKGLADL (SEQ ID NO:4).

In one aspect, provided herein are fusion polypeptides comprising a) at least one immunogenic polypeptide described herein; and b) at least one tumor specific neoantigen.

In one aspect, provided herein are fusion polypeptides comprising a) at least one immunogenic polypeptide described herein; and b) at least one pathogen derived polypeptide. In some embodiments, the at least one pathogen derived polypeptide comprises a viral, bacterial, or parasitic polypeptide.

In one aspect, provided herein are fusion polypeptides comprising: a) immunogenic polypeptide comprising an MHC class II T cell epitope described herein; and b) a SARS-CoV2 spike polypeptide or immunogenic fragment thereof.

In one aspect, provided herein are isolated polynucleotides encoding immunogenic polypeptide comprising an MHC class II T cell epitope described herein, a fusion polypeptide described herein, or a polypeptide described herein. In some embodiments, the polynucleotide is DNA. In some embodiments, the polynucleotide is RNA. In some embodiments, the polynucleotide is mRNA comprising modified ribonucleotides.

In one aspect, provided herein are vectors comprising a polynucleotide described herein.

In one aspect, provided herein are recombinant viruses comprising a polynucleotide described herein.

In one aspect, provided herein are immunogenic composition comprising immunogenic polypeptide comprising an MHC class II T cell epitope described herein, a fusion polypeptide described herein, a polynucleotide described herein, a vector described herein, or a recombinant virus described herein. In some embodiments, the immunogenic composition further comprises an adjuvant.

In one aspect, provided herein are compositions comprising a) at least one immunogenic polypeptide disclosed herein; and b) at least one a target antigen. In some embodiments, the at least one target antigen comprises a hapten. In some embodiments, the at least one target antigen comprises a tumor specific neoantigen. In some embodiments, the at least one target antigen comprises a pathogen derived antigen. In some embodiments, the pathogen derived antigen is a viral, bacterial, or parasitic antigen.

In one aspect, provided herein are pharmaceutical composition comprising immunogenic polypeptide comprising an MHC class II T cell epitope described herein, a fusion polypeptide described herein, a polynucleotide described herein, a vector described herein, a recombinant virus described herein, or a composition described herein and a pharmaceutically acceptable excipient.

In one aspect, provided herein are methods of vaccinating a subject comprising administering a therapeutically effective amount of a pharmaceutical composition described herein or an immunogenic composition described herein to the subject. In some embodiments, the subject is a human.

In one aspect, provided herein are methods of inducing an immune response in a subject comprising administering an effective amount of a pharmaceutical composition described herein or an immunogenic composition described herein to the subject. In some embodiments, the immune response is a viral antigen-specific immune response. In some embodiments, the immune response is a tumor specific immune response.

In one aspect, provided herein are methods of treating a viral infection in a subject comprising administering a therapeutically effective amount of a pharmaceutical composition described herein or an immunogenic composition described herein to the subject. In some embodiments, the viral infection is a SARS-CoV2 infection. In some embodiments, the viral infection is COVID-19.

In one aspect, provided herein are methods of treating a cancer or tumor in a subject comprising administering a therapeutically effective amount of a pharmaceutical composition described herein or an immunogenic composition described herein to the subject.

### BRIEF DESCRIPTION OF THE DRAWINGS

FIG. 1. Low dose human CD4 T-cell responses to individual lumazine synthase peptides. 15-mer level CD4 T-cell responses are shown. Each dot represents the CD4 T-cell response to that peptide from a given subject. Each color indicates a unique subject. One subject was excluded from analysis due to high background in the unstimulated control.

FIG. 2. Low dose cytokine+human CD4 T-cell responses to lumanzine synthase. Positive CD4 T-cell responses by Fisher's exact test are shown. Data displayed is restricted to positive responses. Percent of positive responders for the top 3 peptides is indicated on the graph. Each dot represents the CD4 T-cell response to that peptide from a given subject. Each color indicates a unique subject. One subject was excluded from analysis due to high background in the unstimulated control.

FIG. 3. High dose human CD4 T-cell responses to individual lumazine synthase peptides. 15-mer level CD4 T-cell responses are shown. Each dot represents the CD4 T-cell response to that peptide from a given subject. Each color indicates a unique subject. One subject was excluded from analysis due to high background in the unstimulated control.

FIG. 4. High dose cytokine+ human CD4 T-cell responses to lumanzine synthase. Positive CD4 T-cell responses by Fisher's exact test are shown. Data displayed is restricted to positive responses. Percent of positive responders for the top 3 peptides is indicated on the graph. Each dot represents the CD4 T-cell response to that peptide from a given subject. Each color indicates a unique subject. One subject was excluded from analysis due to high background in the unstimulated control.

### DETAILED DESCRIPTION

In one aspect, provided herein are immunogenic polypeptides comprising one or more MHC class II T cell epitopes capable of activating human CD4 helper T cells in about 90% percent of human PMBC isolates. Without being bound by any particular theory, the immunogenicity of the polypeptides described herein in the majority of human PBMC samples indicates that the polypeptides can be used to improve the immune response elicited by various target antigens, such as a viral antigen, bacterial antigen, parasite antigen and tumor specific neoantigen. Thus, in one aspect, provided herein are immunogenic compositions comprising immunogenic polypeptide comprising an MHC class II T cell epitope described herein and a target antigen. In some embodiments, the target antigen is a viral antigen, bacterial antigen, parasite antigen or tumor specific neoantigen. In some embodiments, the target antigen is a SARS-CoV2 antigen.

### Definitions

Unless defined otherwise, all technical and scientific terms used herein have the same meaning as commonly understood by one of ordinary skill in the art to which this disclosure is related. To facilitate an understanding of the disclosed methods, a number of terms and phrases are defined below.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides described herein are based upon antibodies, in certain embodiments, the polypeptides can occur as single chains or associated chains.

A “fragment” is a portion of a protein or nucleic acid that is substantially identical to a reference protein or nucleic acid. In some embodiments, the portion retains at least 50%, 75%, or 80%, or 90%, 95%, or even 99% of the biological activity of the reference protein or nucleic acid described herein.

The term “immune response” includes T cell mediated and/or B cell mediated immune responses that are influenced by modulation of T cell costimulation. Exemplary immune responses include T cell responses, e.g., cytokine production. In addition, the term immune response includes immune responses that are indirectly affected by T cell activation, e.g., antibody production (humoral responses) and activation of cytokine responsive cells, e.g., macrophages.

The term “MHC class II T cell epitope” refers to a peptide sequence which can be bound by class II MHC molecules in the form of a peptide-presenting MHC molecule or MHC complex and then, in this form, be recognized and bound by CD4 T-helper cells.

An “antigen” is a molecule capable of stimulating an immune response, and can be produced by infectious agents or cancer cells or an autoimmune disease. Antigens recognized by T cells, whether helper T lymphocytes (T helper (TH) cells) or cytotoxic T lymphocytes (CTLs), are not recognized as intact proteins, but rather as small peptides in association with HLA class I or class II proteins on the surface of cells. During the course of a naturally occurring immune response, antigens that are recognized in association with HLA class II molecules on antigen presenting cells (APCs) are acquired from outside the cell, internalized, and processed into small peptides that associate with the HLA class II molecules. APCs can also cross-present peptide antigens by processing exogenous antigens and presenting the processed antigens on HLA class I molecules. Antigens that give rise to peptides that are recognized in association with HLA class I MHC molecules are generally peptides that are produced within the cells, and these antigens are processed and associated with class I MHC molecules. It is now understood that the peptides that associate with given HLA class I or class II molecules are characterized as having a common binding motif, and the binding motifs for a large number of different HLA class I and II molecules have been determined. Synthetic peptides that correspond to the amino acid sequence of a given antigen and that contain a binding motif for a given HLA class I or II molecule can also be synthesized. These peptides can then be added to appropriate APCs, and the APCs can be used to stimulate a T helper cell or CTL response either in vitro or in vivo. Methods for synthesizing the peptides, and methods for stimulating a T helper cell or CTL response are all known and readily available to one of ordinary skill in the art.

The term “neoantigen” refers to a class of tumor antigens which arise from tumor-specific changes in proteins. Neoantigens encompass, but are not limited to, tumor antigens which arise from, for example, a substitution in a protein sequence, a frame shift mutation, a fusion polypeptide, an in-frame deletion, an insertion, and expression of an endogenous retroviral polypeptide. In some embodiments, a neoantigenic peptide or neoantigenic polypeptide comprises a neoepitope.

The terms “linker,” “spacer,” and “hinge” are used interchangeably herein to refer to a peptide or other chemical linkage located between two or more otherwise independent functional domains of an immunogenic composition. For example, a linker may be located between an immunogenic polypeptide and a target antigen. In some embodiments, the linker is a polypeptide located between two domains of a fusion polypeptide, e.g., an immunogenic polypeptide and a target antigen. Suitable linkers for coupling the two or more domains are described herein and/or will otherwise be clear to a person skilled in the art.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, Proc. Natl. Acad. Sci., 87:2264-2268 (1990), as modified in Karlin et al., Proc. Natl. Acad. Sci., 90:5873-5877 (1993), and incorporated into the NBLAST and XBLAST programs (Altschul et al., Nucleic Acids Res., 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology, 266:460-480 (1996)), ALIGN, ALIGN-2 (Genentech, South San Francisco, California) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman (Advances in Applied Mathematics 2: 482 489 (1981)) to find the best segment of homology between two sequences. When using Bestfit or any other sequence alignment program to determine whether a particular sequence is, for instance, 95% identical to a reference sequence described herein, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies described herein do not abrogate the binding of the polypeptide or antibody containing the amino acid sequence, to the antigen(s). Methods of identifying nucleotide and amino acid conservative substitutions which do not eliminate antigen binding are well- known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:.412-417 (1997)).

As used herein, the terms “treatment” or “therapy” (as well as different forms thereof, including curative or palliative) refer to treatment of an infected person. As used herein, the term “treating” includes alleviating or reducing at least one adverse or negative effect or symptom of a condition, disease or disorder. In some embodiments, the condition, disease or disorder is COVID-19. In some embodiments, the condition, disease or disorder is a cancer or tumor.

Terms such as “treating” or “treatment” or “to treat” or “alleviating” or “to alleviate” refer to therapeutic measures that cure, slow down, lessen symptoms of, and/or halt progression of a diagnosed pathologic condition or disorder, such as a viral infection or a tumor or cancer. Thus, those in need of treatment include those already diagnosed with or suspected of having the disorder. In certain embodiments, a subject is successfully “treated” for the disorder according to the methods described herein if the patient shows one or more of the following: a reduction in the number of or complete absence of viral load; a reduction in the viral burden; inhibition of or an absence of the virus into peripheral organs; relief of one or more symptoms associated with the disorder; reduced morbidity and mortality; improvement in quality of life; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof. In some embodiments, the pathologic condition or disorder is COVID-19. In some embodiments, the pathologic condition or disorder is a cancer or tumor.

As used herein, the terms “prevention” or “prophylaxis” refer to preventing a subject from becoming infected with, or reducing the risk of a subject from becoming infected with, or halting transmission of, or the reducing the risk of transmission of a pathogen, e.g., a virus, bacteria, or parasite. In some embodiments, the pathogen is a virus. In some embodiments, the pathogen is SARS-CoV2. Prophylactic or preventative measures refer to measures that prevent and/or slow the development of a targeted pathological condition or disorder. Thus, those in need of prophylactic or preventative measures include those prone to have the disorder and those in whom the disorder is to be prevented.

As employed above and throughout the disclosure the term “effective amount” refers to an amount effective, at dosages, and for periods of time necessary, to achieve the desired result, for example, with respect to the treatment of the relevant disorder, condition, or side effect. An “effective amount” can be determined empirically and in a routine manner, in relation to the stated purpose. It will be appreciated that the effective amount of components of the present invention will vary from subject to subject not only with the particular vaccine, component or composition selected, the route of administration, and the ability of the components to elicit a desired result in the individual, but also with factors such as the disease state or severity of the condition to be alleviated, hormone levels, age, sex, weight of the individual, the state of being of the subject, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

The term “therapeutically effective amount” refers to an amount of a polypeptide, polynucleotide, recombinant virus, immunogenic composition, therapeutic composition, or other drug effective to “treat” a disease or disorder in a subject or mammal. A “prophylactically effective amount” refers to an amount effective, at dosages and for periods of time necessary, to achieve the desired prophylactic result.

The terms “subject,” “individual,” and “patient” are used interchangeably herein, and refer to an animal, for example a human, to whom treatment, including prophylactic treatment, with a immunogenic composition or pharmaceutical composition disclosed herein, is provided. In some embodiments, a subject is a human. In some embodiments, the subject is a non-human animal, for example, a mouse or a cynomolgus monkey. In some embodiments, the subject is a swine, cattle, sheep, goat or rabbit. In some embodiments, the subject is a chicken or turkey.

In one embodiment, the subject, individual, or patient has been infected with a pathogen, e.g., a virus, bacteria or parasite. In one embodiment, the subject, individual, or patient suffers from an infection, e.g., a viral, bacterial or parasitic infection. In one embodiment, the subject, individual, or patient has been exposed to a pathogen, e.g., a virus, bacteria or parasite. In one embodiment, the subject, individual, or patient is at risk of being exposed to a pathogen, e.g., a virus, bacteria or parasite. In one embodiment, the subject, individual, or patient has been infected with a virus, e.g., SARS-CoV2. In one embodiment, the subject, individual, or patient suffers from a viral infection, e.g., COVID-19. In one embodiment, the subject, individual, or patient has been exposed to a virus, e.g., SARS-CoV2. In one embodiment, the subject, individual, or patient is at risk of being exposed to a virus, e.g., SARS-CoV2. In some embodiments, the subject, individual, or patient has a cancer or tumor. In some embodiments, the cancer or tumor is melanoma or glioblastoma. In some embodiments, the cancer or tumor is lung cancer, non-small cell lung cancer, renal cancer, breast cancer, pancreatic cancer, nasopharyngeal cancer, ovarian cancer, cervical cancer, sarcoma, colorectal cancer, HPV16 Associated Cervical Cancer, gastric cancer, or prostate cancer.

The terms “pharmaceutical composition,” “pharmaceutical formulation,” “pharmaceutically acceptable formulation,” or “pharmaceutically acceptable composition” all of which are used interchangeably, refer to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. “Pharmaceutically acceptable” or “pharmaceutical formulation” refers to a preparation which is in such form as to permit the biological activity of the active ingredient to be effective, and which contains no additional components which are unacceptably toxic to a subject to which the formulation would be administered. The formulation can be sterile.

In some embodiments, the term “about” refers to ranges of approximately 10-20% greater than or less than the indicated number or range. In further embodiments, “about” refers to plus or minus 10% of the indicated number or range. For example, “about 10%” indicates a range of 9% to 11%.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed compositions and methods also envisage the explicit exclusion of one or more of any of the group members in the disclosed compositions and methods.

### Immunogenic Polypeptides

In one aspect, provided herein are immunogenic polypeptides comprising an MHC class II T cell epitope, wherein the peptide comprises an amino acid sequence of a fragment of A. aeolicus lumazine synthase. In some embodiments, A. aeolicus lumazine synthase comprises an amino acid sequence that has at least 70%, at least 80%, at least 85%, at least 90%, at least 95%, at least 97%, at least 98% at least 99% or at least 100% identity with MQIYEGKLTAEGLRFGIVASRFNHALVDRLVEGAIDAIVRHGGREEDITLVRVPGSWEIP VAAGELARKEDIDAVIAIGVLIRGATPHFDYIASEVSKGLADLSLELRKPITFGVITADTLE QAIERAGTKHGNKGWEAALSAIEMANLFKSLR (SEQ ID NO:5). In some embodiments, A. aeolicus lumazine synthase comprises the amino acid sequence of SEQ ID NO:5. In some embodiments, the fragment of A. aeolicus lumazine synthase is a fragment of no more than 60, 55, 50, 45, 40, 35, 30, 25, 20, 19, 18, 17, 16, 15, 14, 13, 12, 11, or 10 amino acids. In some embodiments, the fragment is not more than 60, 55, 50, 45, 40, 35, 30, 25, 20, 19, 18, 17, 16, 15, 14, 13, 12, 11, or 10 amino acids long. In some embodiments, the fragment is not more than 60 amino acids long. In some embodiments, the fragment is not more than 55 amino acids long. In some embodiments, the fragment is not more than 50 amino acids long. In some embodiments, the fragment is not more than 45 amino acids long. In some embodiments, the fragment is not more than 40 amino acids long. In some embodiments, the fragment is not more than 35 amino acids long. In some embodiments, the fragment is not more than 30 amino acids long. In some embodiments, the fragment is not more than 25 amino acids long. In some embodiments, the fragment is not more than 20 amino acids long. In some embodiments, the fragment is not more than 15 amino acids long. In some embodiments, the fragment of A. aeolicus lumazine synthase is a fragment consisting of about 60, 55, 50, 45, 40, 35, 30, 25, 20, 19, 18, 17, 16, 15, 14, 13, 12, 11, or 10 amino acids. In some embodiments, the fragment consists of 60 amino acids. In some embodiments, the fragment consists of 55 amino acids. In some embodiments, the fragment consists of 50 amino acids. In some embodiments, the fragment consists of 45 amino acids. In some embodiments, the fragment consists of 40 amino acids. In some embodiments, the fragment consists of 35 amino acids. In some embodiments, the fragment consists of 30 amino acids. In some embodiments, the fragment consists of 25 amino acids. In some embodiments, the fragment consists of 20 amino acids. In some embodiments, the fragment consists of 15 amino acids. In some embodiments, the fragment consists of 14 amino acids. In some embodiments, the fragment consists of 13 amino acids. In some embodiments, the fragment consists of 12 amino acids. In some embodiments, the fragment consists of 11 amino acids. In some embodiments, the fragment consists of 10 amino acids.

In one aspect, provided herein are immunogenic polypeptides comprising one or more MHC class II T cell epitope, wherein the MHC class II T cell epitope comprises an amino acid sequence selected from the group consisting of

(a) 17, 16, 15, 14, 13, 12, 11, or 10
consecutive amino acids of
(SEQ ID NO: 6)
LIRGATPHFDYIASEVSKGLADLSLEL;
(b) 17, 16, 15, 14, 13, 12, 11, or 10
consecutive amino acids of
(SEQ ID NO: 7)
KPITFGVITADTLEQAIERAGTK;
or
(c) 17, 16, 15, 14, 13, 12, 11, or 10
consecutive amino acids of
(SEQ ID NO: 8)
LIRGATPHFDYIASEVSKGLADLSLELRKPITFGVITADTLEQAIERAGT
K.

In some embodiments, the MHC class II T cell epitope comprises an amino acid sequence selected from the group consisting of

(a) 15 consecutive amino acids of
(SEQ ID NO: 6)
LIRGATPHFDYIASEVSKGLADLSLEL;
(b) 15 consecutive amino acids of
(SEQ ID NO: 7)
KPITFGVITADTLEQAIERAGTK;
or
(c) 15 consecutive amino acids of
(SEQ ID NO: 8)
LIRGATPHFDYIASEVSKGLADLSLELRKPITFGVITADTLEQAIERAGT
K.

In some embodiments, the MHC class II T cell epitope comprises an amino acid sequence selected from the group consisting of

(a) 10 consecutive amino acids of
(SEQ ID NO: 6)
LIRGATPHFDYIASEVSKGLADLSLEL;
(b) 10 consecutive amino acids of
(SEQ ID NO: 7)
KPITFGVITADTLEQAIERAGTK;
or
(c) 10 consecutive amino acids of
(SEQ ID NO: 8)
LIRGATPHFDYIASEVSKGLADLSLELRKPITFGVITADTLEQAIERAGT
K.

In some embodiments, the MHC class II T cell epitope comprises an amino acid sequence selected from the group consisting of

(a) 10 consecutive amino acids of
(SEQ ID NO: 1)
ATPHFDYIASEVSKG;
(b) 10 consecutive amino acids of
(SEQ ID NO: 2)
FGVITADTLEQAIER;
or
(c) 10 consecutive amino acids of
(SEQ ID NO: 3)
FDYIASEVSKGLADL.

In one aspect, provided herein are immunogenic polypeptides comprising one or more MHC class II T cell epitope, wherein the MHC class II T cell epitope comprises an amino acid sequence selected from the group consisting of

(SEQ ID NO: 1)
a) ATPHFDYIASEVSKG
(LumSyn22) comprising 0, 1, 2, 3, 4, or 5
substitutions,
(SEQ ID NO: 2)
b) FGVITADTLEQAIER
(LumSyn29) comprising 0, 1, 2, 3, 4, or 5
substitutions,
(SEQ ID NO: 3)
c) FDYIASEVSKGLADL
(LumSyn23) comprising 0, 1, 2, 3, 4, or 5
substitutions,
(SEQ ID NO: 4)
d) ATPHFDYIASEVSKGLADL
(LumSyn22/23) comprising 0, 1, 2, 3, 4,
or 5 substitutions,
e) 9, 10, 11, 12, 13, 14, or 15 consecutive
residues of
(SEQ ID NO: 1)
ATPHFDYIASEVSKG
(LumSyn22),
f) 9, 10, 11, 12, 13, 14, or 15 consecutive
residues of
(SEQ ID NO: 2)
FGVITADTLEQAIER
(LumSyn29),
g) 9, 10, 11, 12, 13, 14, or 15 consecutive
residues of
(SEQ ID NO: 3)
FDYIASEVSKGLADL
(LumSyn23),
and
h) 10, 11, 12, 13, 14, 15, 16, 17, 18, or
19 consecutive residues of
(SEQ ID NO: 4)
ATPHFDYIASEVSKGLADL
(LumSyn22/23).

In some embodiments, the substitutions are conservative substitutions.

In some embodiments, the MHC class II T cell epitope comprises an amino acid sequence selected from the group consisting of a) 9, 10, 11, 12, 13, 14, or 15 consecutive residues of ATPHFDYIASEVSKG (SEQ ID NO:1) (LumSyn22), b) 9, 10, 11, 12, 13, 14, or 15 consecutive residues of FGVITADTLEQAIER (SEQ ID NO:2) (LumSyn29), c) 9, 10, 11, 12, 13, 14, or 15 consecutive residues of FDYIASEVSKGLADL (SEQ ID NO:3) (LumSyn23), and d) 10, 11, 12, 13, 14, 15, 16, 17, 18, or 19 consecutive residues of ATPHFDYIASEVSKGLADL (SEQ ID NO:4) (LumSyn22/23). In some embodiments, the MHC class II T cell epitope comprises an amino acid sequence selected from the group consisting of 9, 10, 11, 12, 13, 14, or 15 consecutive residues of ATPHFDYIASEVSKG (SEQ ID NO:1) (LumSyn22). In some embodiments, the MHC class II T cell epitope comprises an amino acid sequence selected from the group consisting of 9, 10, 11, 12, 13, 14, or 15 consecutive residues of FGVITADTLEQAIER (SEQ ID NO:2) (LumSyn29. In some embodiments, the MHC class II T cell epitope comprises an amino acid sequence selected from the group consisting of 9, 10, 11, 12, 13, 14, or 15 consecutive residues of FDYIASEVSKGLADL (SEQ ID NO:3) (LumSyn23). In some embodiments, the MHC class II T cell epitope comprises an amino acid sequence selected from the group consisting of 10, 11, 12, 13, 14, 15, 16, 17, 18, or 19 consecutive residues of ATPHFDYIASEVSKGLADL (SEQ ID NO:4) (LumSyn22/23). In some embodiments, the MHC class II T cell epitope comprises 9 consecutive residues. In some embodiments, the MHC class II T cell epitope comprises 10 consecutive residues. In some embodiments, the MHC class II T cell epitope comprises 11 consecutive residues. In some embodiments, the MHC class II T cell epitope comprises 12 consecutive residues. In some embodiments, the MHC class II T cell epitope comprises 13 consecutive residues. In some embodiments, the MHC class II T cell epitope comprises 14 consecutive residues. In some embodiments, the MHC class II T cell epitope comprises 15 consecutive residues.

In some embodiments, the MHC class II T cell epitope comprises an amino acid sequence selected from the group consisting of a) ATPHFDYIASEVSKG (SEQ ID NO:1), b) FGVITADTLEQAIER (SEQ ID NO:2), c) FDYIASEVSKGLADL (SEQ ID NO:3), and d) ATPHFDYIASEVSKGLADL (SEQ ID NO:4). In some embodiments, the MHC class II T cell epitope comprises the amino acid sequence of ATPHFDYIASEVSKG (SEQ ID NO:1). In some embodiments, the MHC class II T cell epitope comprises the amino acid sequence of FGVITADTLEQAIER (SEQ ID NO:2). In some embodiments, the MHC class II T cell epitope comprises the amino acid sequence of FDYIASEVSKGLADL (SEQ ID NO:3). In some embodiments, the MHC class II T cell epitope comprises the amino acid sequence of ATPHFDYIASEVSKGLADL (SEQ ID NO:4).

In some embodiments, the immunogenic polypeptide comprises at least 2 MHC class II T cell epitopes. In some embodiments, the at least 2 MHC class II T cell epitopes comprise the amino acid sequences of a) ATPHFDYIASEVSKG (SEQ ID NO:1) (LumSyn22) comprising 0, 1, 2, 3, 4, or 5 substitutions; and b) FGVITADTLEQAIER (SEQ ID NO:2) (LumSyn29) comprising 0, 1, 2, 3, 4, or 5 substitutions. In some embodiments, the at least 2 MHC class II T cell epitopes comprise the amino acid sequences of a) ATPHFDYIASEVSKG (SEQ ID NO:1) (LumSyn22); and b) FGVITADTLEQAIER (SEQ ID NO:2) (LumSyn29). In some embodiments, the at least 2 MHC class II T cell epitopes comprise the amino acid sequences of a) ATPHFDYIASEVSKGLADL (SEQ ID NO:4) (LumSyn22/23) comprising 0, 1, 2, 3, 4, or 5 substitutions; and b) FGVITADTLEQAIER (SEQ ID NO:2) (LumSyn29) comprising 0, 1, 2, 3, 4, or 5 substitutions. In some embodiments, the substitutions are conservative substitutions. In some embodiments, the at least 2 MHC class II T cell epitopes comprise the amino acid sequences of a) ATPHFDYIASEVSKGLADL (SEQ ID NO:4) (LumSyn22/23); and b) FGVITADTLEQAIER (SEQ ID NO:2) (LumSyn29).

In some embodiments, the at least 2 MHC class II T cell epitopes are adjacent to each other in any order.

In some embodiments, the at least 2 MHC class II T cell epitopes are in any order and are separated by a linker peptide. In some embodiments, the linker comprises no more than 10 or no more than 5 amino acid residues. In some embodiments, the linker comprises one or more repeats of the GGS (SEQ ID NO:16) or GGGS (SEQ ID NO:19) sequence. In some embodiments, the linker comprises the amino acid sequence of GGS (SEQ ID NO:16), GGSGGS (SEQ ID NO:17), GGSGGSGGS (SEQ ID NO:18), GGGS (SEQ ID NO:19), GGGSGGGS (SEQ ID NO:20), or GGGSGGGSGGGS (SEQ ID NO:21).

In some embodiments, the immunogenic polypeptide comprises the amino acid sequence of ATPHFDYIASEVSKGLADLSLGGSFGVITADTLEQAIER (SEQ ID NO:9) comprising 0, 1, 2, 3, 4, or 5 substitutions. In some embodiments, the immunogenic polypeptide comprises the amino acid sequence having at least 70%, at least 80%, at least 85%, at least 90%, at least 95%, at least 97%, at least 98% at least 99% or at least 100% identity with ATPHFDYIASEVSKGLADLSLGGSFGVITADTLEQAIER (SEQ ID NO:9). In some embodiments, the immunogenic polypeptide comprises the amino acid sequence of ATPHFDYIASEVSKGLADLSLGGSFGVITADTLEQAIER (SEQ ID NO:9). In some embodiments, the substitutions are conservative substitutions.

In some embodiments, the immunogenic polypeptide comprises the amino acid sequence of ATPHFDYIASEVSKGLADLSLELRKPITFGVITADTLEQAIER (SEQ ID NO:10) comprising 0, 1, 2, 3, 4, or 5 substitutions. In some embodiments, the substitutions are conservative substitutions.

In some embodiments, the immunogenic polypeptide comprises the amino acid sequence having at least 70%, at least 80%, at least 85%, at least 90%, at least 95%, at least 97%, at least 98% at least 99% or at least 100% identity with ATPHFDYIASEVSKGLADLSLELRKPITFGVITADTLEQAIER (SEQ ID NO:10). In some embodiments, the immunogenic polypeptide comprises the amino acid sequence of

(SEQ ID NO: 10)
ATPHFDYIASEVSKGLADLSLELRKPITFGVITADTLEQAIER.

In some embodiments, the immunogenic polypeptide consists of between about 10 and 60, between about 10 and 50, between about 10 and 45, between about 10 and 40, between about 10 and 35, between about 10 and 30, between about 10 and 25, between about 10 and 20, or between about 10 and 15 residues. In some embodiments, the immunogenic polypeptide consists of between about 10 and 60 residues. In some embodiments, the immunogenic polypeptide consists of between about 10 and 50 residues. In some embodiments, the immunogenic polypeptide consists of between about 10 and 45 residues. In some embodiments, the immunogenic polypeptide consists of between about 10 and 40 residues. In some embodiments, the immunogenic polypeptide consists of between about 10 and 35 residues. In some embodiments, the immunogenic polypeptide consists of between about 10 and 30 residues. In some embodiments, the immunogenic polypeptide consists of between about 10 and 25 residues. In some embodiments, the immunogenic polypeptide consists of between about 10 and 20 residues. In some embodiments, the immunogenic polypeptide consists of between about 10 and 15 residues.

In some embodiments, the immunogenic polypeptide is capable of binding to at least one human MHC class II polypeptide. In some embodiments, the immunogenic polypeptide is capable of binding to at least one human HLA-DR MHC class II polypeptide. In some embodiments, the immunogenic polypeptide is capable of binding to at least one human HLA-DP MHC class II polypeptide. In some embodiments, the immunogenic polypeptide is capable of binding to at least one human HLA-DQ MHC class II polypeptide.

In some embodiments, upon contacting human peripheral blood mononuclear cells, the immunogenic polypeptide is capable of inducing IFN gamma, IL-2 and/or CD40L expression in human CD4 T cells. In some embodiments, immunogenic polypeptide is capable of inducing IFN gamma expression. In some embodiments, immunogenic polypeptide is capable of inducing IL-2 expression. In some embodiments, immunogenic polypeptide is capable of inducing CD40L expression. In some embodiments, induction of IFN gamma, IL-2 and/or CD40L expression is detected by Intracellular Cytokine Staining.

Methods to produce the immunogenic polypeptides disclosed herein are known to the skilled artisan. In some embodiments, the immunogenic polypeptides are produced by chemical synthesis. In some embodiments, the immunogenic polypeptides are produced by in vitro translation. In some embodiments, the immunogenic polypeptides are produced by recombinant expression.

### Fusion Polypeptides

In one aspect, provided herein are fusion polypeptides comprising a) at least one immunogenic polypeptide comprising an MHC class II T cell epitope described herein; and b) at least one tumor specific neoantigen polypeptide. Any neoantigen known to a skilled artisan can be used in the context of a immunogenic composition described herein. Neoantigens and methods for identifying neoantigens are disclosed, for example, in US20160339090, US20170199961, US20190307868, US20190151428, and US20200279616, each of which is incorporated herein by reference in its entirety.

In one aspect, provided herein are fusion polypeptides comprising a) at least one immunogenic polypeptide comprising an MHC class II T cell epitope described herein; and b) at least one pathogen derived polypeptide. In some embodiments, the at least one pathogen derived polypeptide comprises a viral, bacterial, or parasitic polypeptide.

In some embodiments, the at least one pathogen derived polypeptide comprises a viral polypeptide. In some embodiments, the viral polypeptide comprises a Betacoronavirus, Human Immunodeficiency Virus (e.g., HIV-Type 1 (HIV-1) and HIV-Type 2 (HIV-2)), Chikungunya virus, Dengue virus, Ebola virus, Eastern Equine Encephalitis virus, Herpes Simplex virus, Human Cytomegalovirus, Human Papillomavirus. Human Metapneumovirus, Influenza virus, Japanese Encephalitis virus, Marburg virus, Measles, Parainfluenza virus, Respiratory Syncytial virus, Sindbis virus, Varicella Zoster virus. Venezuelan Equine Encephalitis virus, West Nile virus, Yellow Fever virus, or Zika virus polypeptide or an immunogenic fragment thereof.

Betacoronavirus. In some embodiments, the viral polypeptide comprises a Betacoronavirus polypeptide or an immunogenic fragment thereof. In some embodiments, the viral polypeptide comprises SARS-CoV2 polypeptide or an immunogenic fragment thereof. In some embodiments, the viral polypeptide comprises MERS-CoV polypeptide or an immunogenic fragment thereof. In some embodiments, the viral polypeptide comprises a SARS-CoV polypeptide or an immunogenic fragment thereof. In some embodiments, the viral polypeptide comprises a Betacoronavirus structural protein. In some embodiments, the viral polypeptide comprises the spike protein (S), envelope protein (E), nucleocapsid protein (N), membrane protein (M) or an immunogenic fragment thereof. In some embodiments, the viral polypeptide comprises a spike protein (S). I. In some embodiments, the viral polypeptide comprises a S1 subunit or a S2 subunit of spike protein (S) or an immunogenic fragment thereof. In some embodiments, the at least one target antigen comprises at least one accessory protein (e.g., protein 3, protein 4a, protein 4b, protein 5), at least one replicase protein (e.g., protein 1a, protein 1b), or a combination of at least one accessory protein and at least one replicase protein.

In some embodiments, the viral polypeptide comprises a MERS-CoV polypeptide, SARS-CoV polypeptide, SARS-CoV2 polypeptide or an immunogenic fragment thereof.

In some embodiments, the viral polypeptide comprises a SARS-CoV2 polypeptide or an immunogenic fragment thereof. In some embodiments, the viral polypeptide comprises a SARS-CoV2 spike protein (S), SARS-CoV2 envelope protein (E), SARS-CoV2 nucleocapsid protein (N), SARS-CoV2 membrane protein (M) or an immunogenic fragment thereof.

In some embodiments, the viral polypeptide comprises a SARS-CoV2 spike protein (S) or an immunogenic fragment thereof. In some embodiments, the SARS-CoV2 spike protein (S) comprises an amino acid sequence having at least 70%, at least 80%, at least 85%, at least 90%, at least 95%, at least 97%, at least 98% at least 99% or at least 100% identity with SQCVNLTTRTQLPPAYTNSFTRGVYYPDKVFRSSVLHSTQDLFLPFFSNVTWFHAIHVSG TNGTKRFDNPVLPFNDGVYFASTEKSNIIRGWIFGTTLDSKTQSLLIVNNATNVVIKVCEF QFCNDPFLGVYYHKNNKSWMESEFRVYSSANNCTFEYVSQPFLMDLEGKQGNFKNLRE FVFKNIDGYFKIYSKHTPINLVRDLPQGFSALEPLVDLPIGINITRFQTLLALHRSYLTPGDS SSGWTAGAAAYYVGYLQPRTFLLKYNENGTITDAVDCALDPLSETKCTLKSFTVEKGIY QTSNFRVQPTESIVRFPNITNLCPFGEVFNATRFASVYAWNRKRISNCVADYSVLYNSASF STFKCYGVSPTKLNDLCFTNVYADSFVIRGDEVRQIAPGQTGKIADYNYKLPDDFTGCVI AWNSNNLDSKVGGNYNYLYRLFRKSNLKPFERDISTEIYQAGSTPCNGVEGFNCYFPLQS YGFQPTNGVGYQPYRVVVLSFELLHAPATVCGPKKSTNLVKNKCVNFNFNGLTGTGVL TESNKKFLPFQQFGRDIADTTDAVRDPQTLEILDITPCSFGGVSVITPGTNTSNQVAVLYQ DVNCTEVPVAIHADQLTPTWRVYSTGSNVFQTRAGCLIGAEHVNNSYECDIPIGAGICAS YQTQTNSPRRARSVASQSIIAYTMSLGAENSVAYSNNSIAIPTNFTISVTTEILPVSMTKTS VDCTMYICGDSTECSNLLLQYGSFCTQLNRALTGIAVEQDKNTQEVFAQVKQIYKTPPIK DFGGFNFSQILPDPSKPSKRSFIEDLLFNKVTLADAGFIKQYGDCLGDIAARDLICAQKFN GLTVLPPLLTDEMIAQYTSALLAGTITSGWTFGAGAALQIPFAMQMAYRFNGIGVTQNV LYENQKLIANQFNSAIGKIQDSLSSTASALGKLQDVVNQNAQALNTLVKQLSSNFGAISS VLNDILSRLDKVEAEVQIDRLITGRLQSLQTYVTQQLIRAAEIRASANLAATKMSECVLG QSKRVDFCGKGYHLMSFPQSAPHGVVFLHVTYVPAQEKNFTTAPAICHDGKAHFPREGV FVSNGTHWFVTQRNFYEPQIITTDNTFVSGNCDVVIGIVNNTVYDPLQPELDSFKEELDK YFKNHTSPDVDLGDISGINASVVNIQKEIDRLNEVAKNLNESLIDLQELGKYEQYIKWPW YIWLGFIAGLIAIVMVTIMLCCMTSCCSCLKGCCSCGSCCKFDEDDSEPVLKGVKLHYT (SEQ ID NO: 11). In some embodiments, the viral polypeptide comprises a trimerized SARS-CoV2 receptor-binding domain. In some embodiments, the viral polypeptide comprises a prefusion stabilized membrane-anchored SARS-CoV2 full-length spike protein. In some embodiments, the viral polypeptide comprises a prefusion stabilized SARS-CoV2 spike protein. Corbett et al., bioRxiv 2020.06.11.145920. doi: 10.1101/2020.06.11.145920.

Human Immunodeficiency Virus (e.g., HIV-Type 1 (HIV-1) and HIV-Type 2 (HIV-2) In some embodiments, the viral polypeptide comprises an Envelope glycoprotein (Env). In some embodiments, the Env is a native Env, an isoform of Env, or a variant of Env (e.g., SOSIP) derived from an HIV isolate. In some embodiments, the Env is a well-ordered Env trimer. In one embodiment, a well-ordered Env trimer is a native flexibly linked (NFL) trimer as described in Sharna, et al., Cell Reports, 11(4):539-50 (2015). In one embodiment, a well-ordered Env trimer is a DS-SOSIP as described in Chuang, et al., J. Virology, 91(10). pii: e02268-16 (2017).

Chikungunya Virus. In some embodiments, the viral polypeptide comprises a CHIKV structural protein selected from an envelope protein (E) (e.g., E1, E2, E3), a 6K protein, or a capsid (C) protein.

Dengue virus. In some embodiments, the viral polypeptide comprises a DENV capsid protein, a DENV membrane protein, a DENV precursor-membrane protein, a DENV precursor membrane (pry) and envelope (E) polypeptide (DENY prME), or a DENV non-structural protein selected from NS1, NS2A, NS2B, NS3, NS4A, NS4B, and NS5. In some embodiments, the viral polypeptide comprises a polypeptide is from a DENV serotype selected from DENV-1, DENY-2, DENV-3, DENY-4, and DENY-5.

Ebola virus. In some embodiments, the viral polypeptide comprises an EBOV glycoprotein (GP), surface EBOV GP, wild type EBOV pro-GP, mature EBOV GP, secreted wild type EBOV pro-GP, secreted mature EBOV GP, EBOV nucleoprotein (NP), RNA polymerase L, and EBOV matrix protein selected from VP35, VP40, VP24, or VP30.

Herpes Simpler virus. In some embodiments, the viral polypeptide comprises HSV (HSV-1 or HSV-2) glycoprotein B, HSV (HSV-1 or HSV-2) glycoprotein C, HSV (HSV-1 or HSV-2) glycoprotein D, HSV (HSV-1 or HSV-2) glycoprotein E, or HSV (HSV-1 or HSV-2) glycoprotein I.

Human Cytomegalovirus. In some embodiments, the viral polypeptide comprises a HCMV gH, gL, gB, gO, gN, gM, UL83, UL123, UL128, UL130, or UL131A protein.

Human Papillomavirus. In some embodiments, the viral polypeptide comprises an HPV E1, E2, E4, E5, E6, E7, L1, or L2 protein, e.g., obtained from HPV serotypes 6, 11, 16, 18, 31, 33, 35, 39, 45, 51, 52, 56, 58, 59, 68, 73 or 82.

Human Metapneumovirus, Parainfluenza virus and Respiratory Syncytial virus. In some embodiments, the viral polypeptide comprises a major surface glycoprotein G or an immunogenic fragment thereof. In some embodiments, the viral polypeptide comprises a Fusion (F) glycoprotein (e.g., Fusion glycoprotein F0, F1 or F2) or an immunogenic fragment thereof. In some embodiments, the viral polypeptide comprises a major surface glycoprotein G or an immunogenic fragment thereof and F glycoprotein or an immunogenic fragment thereof. In some embodiments, the viral polypeptide comprises a nucleoprotein (N), phosphoprotein (P), large polymerase protein (L), matrix protein (M), small hydrophobic protein (SH), nonstructural protein 1 (NS1), nonstructural protein 2 (NS2) or an immunogenic fragment thereof.

Influenza virus. In some embodiments, the viral polypeptide comprises an antigenic subdomain of HA, termed HA1, HA2, or a combination of HA1 and HA2 (or a combination of both, of any one of or a combination of any or all of H2, H3, H4, H5, H6, H7, H8, H9, H10, H11, H12, H13, H14, H15, H16, H17, and/or H18). In some embodiments, the viral polypeptide comprises a neuraminidase (NA). In some embodiments, the viral polypeptide comprises nucleoprotein (NP), matrix protein 1 (M1), matrix protein 2 (M2), non-structural protein 1 (NS1) or non-structural protein 2 (NS2).

Japanese Encephalitis virus. In some embodiments, the viral polypeptide comprises JEV E protein, JEV Es, JEV prM, JEV capsid, JEV NS1, or JEV prM and E polyprotein (prME).

Marburg virus. In some embodiments, the viral polypeptide comprises a MARV glycoprotein (GP).

Measles. In some embodiments, the viral polypeptide comprises a hemagglutinin (HA) protein or an immunogenic fragment thereof. In some embodiments, the viral polypeptide comprises a Fusion (F) protein or an immunogenic fragment thereof. In some embodiments, the viral polypeptide is from MeV strain D3 or B8, for example

Varicella Zoster virus. In some embodiments, the viral polypeptide comprises a VZV glycoprotein selected from VZV gE, gI, gB, gH, gK, gL, gC, gN, and gM.

West Nile virus, Eastern Equine Encephalitis virus, Venezuelan Equine Encephalitis virus, and Sindbis virus. In some embodiments, the viral polypeptide comprises at least one Arbovirus antigen and/or at least one Alphavirus antigen.

Yellow Fever virus. In some embodiments, the viral polypeptide comprises a YFV polyprotein, a YFV capsid protein, a YFV premembrane/membrane protein, a YFV envelope protein, a YFV non-structural protein 1, a YFV non-structural protein 2A, a YFV non-structural protein 2B, a YFV non-structural protein 3, a YFV non-structural protein 4A, a YFV non-structural protein 4B, or a YFV non-structural protein 5.

Zika virus antigens. In some embodiments, the viral polypeptide comprises a ZIKV polyprotein, a ZIKV capsid protein, a ZIKV premembrane/membrane protein, a ZIKV envelope protein, a ZIKV non-structural protein 1, a ZIKV non-structural protein 2A, a ZIKV n on-structural protein 213, a ZIKV non-structural protein 3, a ZIKV non-structural protein 4A, a ZIKV non-structural protein 4B, or a ZIKV non-structural protein 5.

In some embodiments, the at least one pathogen derived polypeptide comprises a bacterial polypeptide. Examples of bacterial polypeptides include, but are not limited to, Chlamydia trachomatis polypeptides, Lyme Borrelia polypeptides and Streptococcal polypeptides. In some embodiments, the bacterial polypeptide comprises a major outer membrane protein (MOMP or OmpA), e.g., from Chlamydia trachomatis serovar (serotype) H, F, E, D, I, G, J or K. In some embodiments, the bacterial polypeptide comprises a Borrelia OspA protein.

In some embodiments, the at least one pathogen derived polypeptide comprises a parasitic polypeptide. Examples of parasitic polypeptides include, but are not limited to, Plasmodium falciparum, Plasmodium vivax, Plasmodium ovale, and Plasmodium malariae, and Plasmodium knowlesi polypeptides. In some embodiments, the parasitic polypeptide comprises a circumsporozoite (CS) protein or an immunogenic fragment thereof. In some embodiments, the parasitic polypeptide comprises a RTS hybrid protein. In some embodiments, the parasitic polypeptide comprises a merozoite surface protein-1 (MSP1), apical membrane antigen 1 (AMA1), thrombospondin related adhesive protein (TRAP) or an immunogenic fragment thereof.

In some embodiments, the at least one immunogenic polypeptide and the at least one pathogen derived polypeptide are directly linked in any order.

In some embodiments, the at least one immunogenic polypeptide and the at least one pathogen derived polypeptide are separated by a linker. In some embodiments, the linker comprises no more than 10 amino acid residues. In some embodiments, the linker comprises no more than 5 amino acid residues. In some embodiments, the linker comprises one or more repeats of the GGS (SEQ ID NO:16) or GGGS (SEQ ID NO:19) sequence. In some embodiments, the linker comprises the amino acid sequence of GGS (SEQ ID NO:16), GGSGGS (SEQ ID NO:17), GGSGGSGGS (SEQ ID NO:18), GGGS (SEQ ID NO:19), GGGSGGGS (SEQ ID NO:20), or GGGSGGGSGGGS (SEQ ID NO:21).

In one aspect, provided herein are fusion polypeptides comprising: a) immunogenic polypeptide comprising an MHC class II T cell epitope described herein; and b) a SARS-CoV2 spike polypeptide or immunogenic fragment thereof. In some embodiments, the fusion polypeptide further comprises a transmembrane domain. In some embodiments, the fusion polypeptide further comprises at least one linker.

In some embodiments, the immunogenic polypeptide comprises at least one amino acid sequence selected from the group consisting of a) ATPHFDYIASEVSKG (SEQ ID NO:1) comprising 0, 1, 2, 3, 4, or 5 substitutions, b) FGVITADTLEQAIER (SEQ ID NO:2) comprising 0, 1, 2, 3, 4, or 5 substitutions, c) FDYIASEVSKGLADL (SEQ ID NO:3) comprising 0, 1, 2, 3, 4, or 5 substitutions, and d) ATPHFDYIASEVSKGLADL (SEQ ID NO:4) comprising 0, 1, 2, 3, 4, or 5 substitutions. In some embodiments, the immunogenic polypeptide comprises at least one amino acid sequence selected from the group consisting of a) ATPHFDYIASEVSKG (SEQ ID NO:1), b) FGVITADTLEQAIER (SEQ ID NO:2), c) FDYIASEVSKGLADL (SEQ ID NO:3), and d) ATPHFDYIASEVSKGLADL (SEQ ID NO:4). In some embodiments, the immunogenic polypeptide comprises the amino acid sequences of ATPHFDYIASEVSKGLADL (SEQ ID NO:4) (LumSyn22/23); and FGVITADTLEQAIER (SEQ ID NO:2) (LumSyn29). In some embodiments, the immunogenic polypeptide further comprises a linker. In some embodiments, the immunogenic polypeptide comprises the amino acid sequence of ATPHFDYIASEVSKGLADLGGSFGVITADTLEQAIER (SEQ ID NO:12). In some embodiments, the immunogenic polypeptide comprises the amino acid sequence of

(SEQ ID NO: 9)
ATPHFDYIASEVSKGLADLSLGGSFGVITADTLEQAIER.

In some embodiments, the SARS-CoV2 spike polypeptide or immunogenic fragment thereof comprises the S1 subunit. In some embodiments, the SARS-CoV2 spike polypeptide or immunogenic fragment thereof comprises the S2 subunit. In some embodiments, the SARS-CoV2 spike polypeptide or immunogenic fragment thereof comprises the receptor binding domain of the SARS-CoV2 spike protein. Huang et al., Acta Pharmacologica Sinica 41(9):1141-1149 (2020).

In some embodiments, the receptor binding domain comprises an amino acid sequence having at least 70%, at least 80%, at least 85%, at least 90%, at least 95%, at least 97%, at least 98% at least 99% or at least 100% identity with TNLCPFGEVFNATRFASVYAWNRKRISNCVADYSVLYNSASFSTFKCYGVSPTKLNDLC FTNVYADSFVIRGDEVRQIAPGQTGKIADYNYKLPDDFTGCVIAWNSNNLDSKVGGNYN YLYRLFRKSNLKPFERDISTEIYQAGSTPCNGVEGFNCYFPLQSYGFQPTNGVGYQPYRV VVLSFELLHAPATVCGP (SEQ ID NO: 13). In some embodiments, the receptor binding domain comprises the amino acid sequence of SEQ ID NO: 13.

In some embodiments, the fusion polypeptide further comprises a transmembrane domain. In some embodiments, the transmembrane domain comprises the Vesicular Stomatitis Virus G* glycoprotein (UniProtKB/Swiss-Prot: Q8B0H6.1) transmembrane domain. In some embodiments, the transmembrane domain comprises the amino acid sequence of

(SEQ ID NO: 15)
KSSIASFFFIIGLIIGLFLVLR.

In some embodiments, the fusion polypeptide comprises the structure of SP-IP-TM, wherein SP denotes the SARS-CoV2 spike polypeptide or immunogenic fragment thereof, IP denotes the immunogenic polypeptide, and TM denotes the transmembrane domain.

In some embodiments, the fusion polypeptide comprises the structure of SP-L1-IP -L2-TM, wherein SP denotes the SARS-CoV2 spike polypeptide or immunogenic fragment thereof, IP denotes the immunogenic polypeptide, TM denotes the transmembrane domain, and L1 and L2 denote a linker polypeptide. In some embodiments, the L1 and L2 linkers comprise the same sequence. In some embodiments, the L1 and L2 linkers comprise different sequences. In some embodiments, the L1 and L2 linkers comprise between about 5 and 15 amino acid sequences. In some embodiments, the L1 and L2 linkers comprise the amino acid sequence of

(SEQ ID NO: 21)
GGGSGGGSGGGS.

In some embodiments, the fusion polypeptide comprises an amino acid sequence having at least 70%, at least 80%, at least 85%, at least 90%, at least 95%, at least 97%, at least 98% at least 99% or at least 100% identity with

(SEQ ID NO: 14)
TNLCPFGEVFNATRFASVYAWNRKRISNCVADYSVLYNSASFSTFKCYGV
SPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNG
VEGFNCYFPLQSYGFQPTNGVGYQPYRVVVLSFELLHAPATVCGPGGSGG
SGGSATPHFDYIASEVSKGLADLSLGGSFDYIASEVSKGLADLGGSGGSG
GSKSSIASFFFIIGLIIGLFLVLR.

In some embodiments, the fusion polypeptide comprises the amino acid sequence of SEQ ID NO:14.

In some embodiments, the fusion polypeptide further comprises a signal sequence.

### Polynucleotides

In one aspect, provided herein are isolated polynucleotides encoding immunogenic polypeptide comprising an MHC class II T cell epitope described herein, a fusion polypeptide described herein, or a polypeptide described herein.

In some embodiments, the polynucleotide is DNA.

In some embodiments, the polynucleotide is RNA. In some embodiments, the polynucleotide is mRNA. In some embodiment the RNA, e.g., mRNA comprises modified ribonucleotides.

In some embodiments, an mRNA disclosed herein comprises a coding region encoding a polypeptide disclosed herein, and additionally comprises one or more of a 5′ untranslated region, 3′ untranslated region, 5′ cap, and polyadenylation signal. In some embodiments, an mRNA disclosed herein comprises a coding region encoding a polypeptide disclosed herein, a 5′ untranslated region, a 3′ untranslated region, a 5′ cap, and a polyadenylation signal. In some embodiments, an mRNA disclosed herein comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

In some embodiments, a polynucleotide disclosed herein encodes a fusion polypeptide comprising: a) immunogenic polypeptide comprising an MHC class II T cell epitope described herein; and b) a SARS-CoV2 spike polypeptide or immunogenic fragment thereof. In some embodiments, the immunogenic polypeptide comprises the amino acid sequences of ATPHFDYIASEVSKGLADL (SEQ ID NO:4) (LumSyn22/23); and FGVITADTLEQAIER (SEQ ID NO:2) (LumSyn29). In some embodiments, the immunogenic polypeptide comprises the amino acid sequence of ATPHFDYIASEVSKGLADLSLGGSFGVITADTLEQAIER (SEQ ID NO:9). In some embodiments, the SARS-CoV2 spike polypeptide or immunogenic fragment thereof comprises the receptor binding domain of the SARS-CoV2 spike protein. In some embodiments, the fusion polypeptide further comprises a transmembrane domain. In some embodiments, the transmembrane domain comprises the amino acid sequence of KSSIASFFFIIGLIIGLFLVLR (SEQ ID NO:15). In some embodiments, the fusion polypeptide comprises the structure of SP-L1-IP-L2-TM, wherein SP denotes the SARS-CoV2 spike polypeptide or immunogenic fragment thereof, IP denotes the immunogenic polypeptide, TM denotes the transmembrane domain, and L1 and L2 denote a linker polypeptide. In some embodiments, the fusion polypeptide comprises an amino acid sequence having at least 70%, at least 80%, at least 85%, at least 90%, at least 95%, at least 97%, at least 98% at least 99% or at least 100% identity with SEQ ID NO:14. In some embodiments, the polynucleotide is a DNA. In some embodiments, the polynucleotide is an RNA. In some embodiment the RNA, e.g., mRNA comprises modified ribonucleotides. In some embodiments, an mRNA disclosed herein comprises a coding region encoding a polypeptide disclosed herein, and additionally comprises one or more of a 5′ untranslated region, 3′ untranslated region, 5′ cap, and polyadenylation signal. In some embodiments, an mRNA disclosed herein comprises a coding region encoding a polypeptide disclosed herein, a 5′ untranslated region, a 3′ untranslated region, a 5′ cap, and a polyadenylation signal. In some embodiments, an mRNA disclosed herein comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

In one aspect, provided herein are vectors comprising a polynucleotide described herein.

In one aspect, provided herein are recombinant viruses comprising a polynucleotide described herein.

### Immunogenic Compositions

In one aspect, provided herein are immunogenic compositions comprising an immunogenic polypeptide comprising an MHC class II T cell epitope described herein, a fusion polypeptide described herein, a polynucleotide described herein, a vector described herein, or a recombinant virus described herein. In some embodiments, the immunogenic composition further comprises an adjuvant. In some embodiments, the immunogenic composition comprises an immunogenic polypeptide comprising an MHC class II T cell epitope described herein. In some embodiments, the immunogenic composition comprises a fusion polypeptide described herein. In some embodiments, the immunogenic composition comprises a polynucleotide described herein. In some embodiments, the immunogenic composition comprises a vector described herein. In some embodiments, the immunogenic composition comprises a recombinant virus described herein.

In some embodiments, an immunogenic composition described herein comprises an mRNA encoding a fusion polypeptide comprising an immunogenic polypeptide described herein. In some embodiments, the immunogenic composition comprises a cationic lipid nanoparticle (LNP) encapsulating mRNA having an open reading frame encoding at least one target antigen, an immunogenic peptide described herein, and a 5′ terminal cap modified to increase mRNA translation efficiency. In some embodiments, the cationic lipid nanoparticle comprises a cationic lipid, a PEG-modified lipid, a sterol and a non-cationic lipid. See, e.g., US20200261572, US20190351040, and US20190211065, each of which is incorporated herein by reference in its entirety.

In some embodiments, an immunogenic composition comprises a polynucleotide encoding a fusion polypeptide comprising: a) immunogenic polypeptide comprising an MHC class II T cell epitope described herein; and b) a SARS-CoV2 spike polypeptide or immunogenic fragment thereof. In some embodiments, the fusion polypeptide comprises an amino acid sequence having at least 70%, at least 80%, at least 85%, at least 90%, at least 95%, at least 97%, at least 98% at least 99% or at least 100% identity with SEQ ID NO:14.

In one aspect, provided herein are compositions comprising a) at least one immunogenic polypeptide disclosed herein; and b) at least one a target antigen. In some embodiments, the at least one target antigen comprises a hapten. In some embodiments, the at least one target antigen comprises a tumor specific neoantigen. In some embodiments, the at least one target antigen comprises a pathogen derived antigen. In some embodiments, the pathogen derived antigen is a viral, bacterial, or parasitic antigen.

In some embodiments, the target antigen comprises a viral, bacterial, or parasitic polypeptide or antigenic fragment thereof.

In some embodiments, the target antigen is a viral antigen. A “viral antigen” is an antigen encoded by a viral genome. Examples of viral antigens include, but are not limited to, Betacoronavirus, Human Immunodeficiency Virus (e.g., HIV-Type 1 (HIV-1) and HIV-Type 2 (HIV-2)), Chikungunya virus, Dengue virus, Ebola virus, Eastern Equine Encephalitis virus, Herpes Simplex virus, Human Cytomegalovirus, Human Papillomavirus, Human Metapneumovirus, Influenza virus, Japanese Encephalitis virus, Marburg virus, Measles, Parainfluenza virus, Respiratory Syncytial virus, Sindbis virus, Varicella Zoster virus, Venezuelan Equine Encephalitis virus, West Nile virus, Yellow Fever virus, and Zika virus antigens.

In some embodiments, the target antigen comprises a viral polypeptide of an antigenic fragment thereof. In some embodiments, the viral polypeptide is selected from the group consisting of Betacoronavirus, Human Immunodeficiency Virus (e.g., HIV-Type 1 (HIV-1) and HIV-Type 2 (HIV-2), Chikungunya virus, Dengue virus, Ebola virus, Eastern Equine Encephalitis virus, Herpes Simplex virus, Human Cytomegalovirus, Human Papillomavirus. Human Metapneumovirus, Influenza virus, Japanese Encephalitis virus, Marburg virus, Measles, Parainfluenza virus, Respiratory Syncytial virus, Sindbis virus, Varicella Zoster virus. Venezuelan Equine Encephalitis virus, West Nile virus, Yellow Fever virus, or Zika virus polypeptide and an immunogenic fragment thereof.

Betacoronavirus. In some embodiments, the BetaCoV is SARS-CoV2. In some embodiments, the BetaCoV is MERS-CoV. In some embodiments, the BetaCoV is SARS-CoV. In some embodiments, the at least one target antigen comprises a Betacoronavirus structural protein. . In some embodiments, the at least one target antigen comprises the spike protein (S), envelope protein (E), nucleocapsid protein (N), membrane protein (M) or an immunogenic fragment thereof. In some embodiments, the at least one target antigen comprises a spike protein (S). I. In some embodiments, the at least one target antigen comprises a S1 subunit or a S2 subunit of spike protein (S) or an immunogenic fragment thereof. In some embodiments, the at least one target antigen comprises at least one accessory protein (e.g., protein 3, protein 4a, protein 4b, protein 5), at least one replicase protein (e.g., protein 1a, protein 1b), or a combination of at least one accessory protein and at least one replicase protein.

In some embodiments, the viral polypeptide is selected from the group consisting of MERS-CoV polypeptide, SARS-CoV polypeptide, SARS-CoV2 polypeptide and an immunogenic fragment thereof.

In some embodiments, the viral polypeptide comprises a SARS-CoV2 polypeptide or an immunogenic fragment thereof. In some embodiments, the viral polypeptide comprises a SARS-CoV2 spike protein (S), SARS-CoV2 envelope protein (E), SARS-CoV2 nucleocapsid protein (N), SARS-CoV2 membrane protein (M) or an immunogenic fragment thereof.

In some embodiments, the viral polypeptide comprises a SARS-CoV2 spike protein (S) or an immunogenic fragment thereof. In some embodiments, the SARS-CoV2 spike protein (S) comprises an amino acid sequence having at least 70%, at least 80%, at least 85%, at least 90%, at least 95%, at least 97%, at least 98% at least 99% or at least 100% identity with SEQ ID NO: 11.

Human Immunodeficiency Virus (e.g., HIV-Type 1 (HIV-1) and HIV-Type 2 (HIV-2). In some embodiments, the at least one target antigen comprises an Envelope glycoprotein (Env). In some embodiments, the Env is a native Env, an isoform of Env, or a variant of Env (e.g., SOSIP) derived from an HIV isolate. In some embodiments, the Env is a well-ordered Env trimer. In one embodiment, a well-ordered Env trimer is a native flexibly linked (NFL) trimer as described in Sharna, et al., Cell Reports, 11(4):539-50 (2015). In one embodiment, a well-ordered Env trimer is a DS-SOSIP as described in Chuang, et al., J. Virology, 91(10). pii: e02268-16 (2017).

Chikungunya Virus. In some embodiments, the at least one target antigen comprises a CHIKV structural protein selected from an envelope protein (E) (e.g., E1, E2, E3), a 6K protein, or a capsid (C) protein.

Dengue virus. In some embodiments, the at least one target antigen comprises a DENV capsid protein, a DENV membrane protein, a DENV precursor-membrane protein, a DENV precursor membrane (pry) and envelope (E) polypeptide (DENY prME), or a DENV non-structural protein selected from NS1, NS2A, NS2B, NS3, NS4A, NS4B, and NSS. In some embodiments, the at least one target antigen comprises a polypeptide is from a DENV serotype selected from DENV-1, DENY-2, DENV-3, DENY-4, and DENY-5.

Ebola virus. In some embodiments, the at least one target antigen comprises an EBOV glycoprotein (GP), surface EBOV GP, wild type EBOV pro-GP, mature EBOV GP, secreted wild type EBOV pro-GP, secreted mature EBOV GP, EBOV nucleoprotein (NP), RNA polymerase L, and EBOV matrix protein selected from VP35, VP40, VP24, or VP30.

Herpes Simpler virus. In some embodiments, the at least one target antigen comprises HSV (HSV-1 or HSV-2) glycoprotein B, HSV (HSV-1 or HSV-2) glycoprotein C, HSV (HSV-1 or HSV-2) glycoprotein D, HSV (HSV-1 or HSV-2) glycoprotein E, or HSV (HSV-1 or HSV-2) glycoprotein I.

Human Cytomegalovirus. In some embodiments, the at least one target antigen comprises a HCMV gH, gL, gB, gO, gN, gM, UL83, UL123, UL128, UL130, or UL131A protein.

Human Papillomavirus. In some embodiments, the at least one target antigen comprises an HPV E1, E2, E4, E5, E6, E7, L1, or L2 protein, e.g., obtained from HPV serotypes 6, 11, 16, 18, 31, 33, 35, 39, 45, 51, 52, 56, 58, 59, 68, 73 or 82.

Human Metapneumovirus, Parainfluenza virus and Respiratory Syncytial virus. In some embodiments, the at least one target antigen comprises a major surface glycoprotein G or an immunogenic fragment thereof. In some embodiments, the at least one antigen comprises a Fusion (F) glycoprotein (e.g., Fusion glycoprotein F0, F1 or F2) or an immunogenic fragment thereof. In some embodiments, the at least one antigen comprises a major surface glycoprotein G or an immunogenic fragment thereof and F glycoprotein or an immunogenic fragment thereof. In some embodiments, the at least one antigen comprises a nucleoprotein (N), phosphoprotein (P), large polymerase protein (L), matrix protein (M), small hydrophobic protein (SH), nonstructural protein 1 (NS1), nonstructural protein 2 (NS2) or an immunogenic fragment thereof.

Influenza virus. In some embodiments, the at least one target antigen comprises an antigenic subdomain of HA, termed HA1, HA2, or a combination of HA1 and HA2 (or a combination of both, of any one of or a combination of any or all of H2, H3, H4, H5, H6, H7, H8, H9, H10, H11, H12, H13, H14, H15, H16, H17, and/or H18). In some embodiments, the at least one target antigen comprises a neuraminidase (NA). In some embodiments, the at least one target antigen comprises nucleoprotein (NP), matrix protein 1 (M1), matrix protein 2 (M2), non-structural protein 1 (NS1) or non-structural protein 2 (NS2).

Japanese Encephalitis virus. In some embodiments, the at least one target antigen comprises JEV E protein, JEV Es, JEV prM, JEV capsid, JEV NS1, or JEV prM and E polyprotein (prME).

Marburg virus. In some embodiments, the at least one target antigen comprises a MARV glycoprotein (GP).

Measles. In some embodiments, the at least one target antigen comprises a hemagglutinin (HA) protein or an immunogenic fragment thereof. In some embodiments, the at least one target antigen comprises a Fusion (F) protein or an immunogenic fragment thereof. In some embodiments, the at least one antigen is from MeV strain D3 or B8, for example.

Varicella Zoster virus. In some embodiments, the at least one target antigen comprises a VZV glycoprotein selected from VZV gE, gI, gB, gH, gK, gL, gC, gN, and gM.

West Nile virus, Eastern Equine Encephalitis virus, Venezuelan Equine Encephalitis virus, and Sindbis virus. In some embodiments, the at least one target antigen comprises at least one Arbovirus antigen and/or at least one Alphavirus antigen.

Yellow Fever virus. In some embodiments, the at least one target antigen comprises a YFV polyprotein, a YFV capsid protein, a YFV premembrane/membrane protein, a YFV envelope protein, a YFV non-structural protein 1, a YFV non-structural protein 2A, a YFV non-structural protein 2B, a YFV non-structural protein 3, a YFV non-structural protein 4A, a YFV non-structural protein 4B, or a YFV non-structural protein 5.

Zika virus antigens. In some embodiments, the at least one target antigen comprises a ZIKV polyprotein, a ZIKV capsid protein, a ZIKV premembrane/membrane protein, a ZIKV envelope protein, a ZIKV non-structural protein 1, a ZIKV non-structural protein 2A, a ZIKV non-structural protein 213, a ZIKV non-structural protein 3, a ZIKV non-structural protein 4A, a ZIKV non-structural protein 4B, or a ZIKV non-structural protein 5.

In some embodiments, the target antigen is a bacterial antigen. A bacterial antigen is an antigen encoded by a bacterial genome. In some embodiments, an immunogenic composition of described herein comprises a bacterial polypeptide antigen. Examples of bacterial antigens include, but are not limited to, Chlamydia trachomatis antigen, Lyme Borrelia antigen and Streptococcal antigen. In some embodiments, the at least one target antigen comprises a major outer membrane protein (MOMP or OmpA), e.g., from Chlamydia trachomatis serovar (serotype) H, F, E, D, I, G, J or K. In some embodiments, the at least one target antigen comprises a Borrelia OspA protein.

In some embodiments, the target antigen is a parasitic antigen. A parasitic antigen is an antigen encoded by a parasitic genome. In some embodiments, an immunogenic composition described herein comprises a parasitic polypeptide antigen. Examples of parasitic antigens include, but are not limited to, Plasmodium falciparum, Plasmodium vivax, Plasmodium ovale, and Plasmodium malariae, and Plasmodium knowlesi antigens. In some embodiments, the at least one target antigen comprises a circumsporozoite (CS) protein or an immunogenic fragment thereof. In some embodiments, the at least one target antigen comprises a RTS hybrid protein. In some embodiments, the at least one target antigen comprises a merozoite surface protein-1 (MSP1), apical membrane antigen 1 (AMA1), thrombospondin related adhesive protein (TRAP) or an immunogenic fragment thereof.

In some embodiments, the target antigen is a tumor specific neoantigen. Any neoantigen known to a skilled artisan can be used in the context of an immunogenic composition described herein. Neoantigens are disclosed, for example, in US20160339090, US20170199961, US20190307868, US20190151428, and US20200279616, each of which is incorporated herein by reference in its entirety.

In some embodiments, the at least one immunogenic polypeptide and at least one the target antigen are covalently linked.

In some embodiments, an immunogenic composition described herein further comprises an adjuvant. Suitable adjuvants are known in the art. Suitable adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538- 44; Ahmad-Nejad, P. et al (2002) Eur. J. Immunol. 32(7): 1958-68; poly IC or poly AU acids, polyarginine with or without CpG (also known in the art as IC3 1; see Schellack, C. et al (2003) Proceedings of the 34th Annual Meeting of the German Society of Immunology; Lingnau, K. et al (2002) Vaccine 20(29-30): 3498-508), JuvaVax™ (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-0-acylated monophosphoryl lipid A (3D-MPL), imiquimod (also known in the art as IQM and commercially available as Aldara®; U.S. Pat. Nos. 4,689,338; 5,238,944; Zuber, A K. et al (2004) 22(13-14): 1791-8), and the CCRS inhibitor CMPD167 (see Veazey, R S. et al (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A M. et al (2001) J. Immunol. 167(6): 3398-405), polyphosphazenes (Allcock, H. R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IL-15 IGF-1, IFN-a, IFN- , and IFN-y (Boyer et al., (2002) J. Liposome Res. 121:137-142; WOO1/095919), immunoregulatory proteins such as CD40L (ADX40; see, for example, W003/063899), and the CD1a ligand of natural killer cells (also known as CRONY or a-galactosyl ceramide; see Green, T. D. et al, (2003) J. Virol. 77(3): 2046-2055), immunostimulatory fusion proteins such as IL-2 fused to the Fe fragment of immunoglobulins (Barouch et al., Science 290:486-492, 2000) and co-stimulatory molecules B7. 1 and B7.2 (Boyer), all of which can be administered either as proteins or in the form of DNA, on the same expression vectors as those encoding the antigens described herein or on separate expression vectors. In some embodiments, the adjuvant comprises lecithin combined with an acrylic polymer (Adjuplex-LAP), lecithin coated oil droplets in an oil-in-water emulsion (Adjuplex-LE) or lecithin and acrylic polymer in an oil-in-water emulsion (Adjuplex-LAO) (Advanced BioAdjuvants (ABA)). In some embodiments, the adjuvant comprises lecithin. In some embodiments, the adjuvant comprises alum. In some embodiments, the adjuvant comprises saponin, cholesterol and phospholipid. In some embodiments, the adjuvant comprises ISCOMATRIX™. In some embodiments, the adjuvant comprises carbomer homopolymer and lecithin. In some embodiments, the adjuvant comprises Adjuplex™. In some embodiments, the adjuvant comprises poly-ICLC or poly(I:C). In some embodiments, the adjuvant can be a mixture of emulsifier(s), micelle-forming agent, and oil such as that which is commercially available under the name Provax® (IDEC Pharmaceuticals, San Diego, CA). (PEG).

In some embodiments, an immunogenic composition described herein is capable of eliciting an increased immune response in a subject compared to the immune response elicited by a reference immunogenic composition not comprising the polypeptide comprising an MHC class II T cell epitope described herein. In some embodiments, the increased immune response is an increased humoral response. In some embodiments, the increased immune response is an increased cellular immune response. In some embodiments, the subject is a mouse or a cynomolgus monkey.

### Pharmaceutical Compositions

In one aspect, provided herein are pharmaceutical composition comprising immunogenic polypeptide comprising an MHC class II T cell epitope described herein, a fusion polypeptide described herein, a polynucleotide described herein, a vector described herein, a recombinant virus described herein, or a composition described herein and a pharmaceutically acceptable excipient. In some embodiments, the pharmaceutical composition comprises an immunogenic polypeptide comprising an MHC class II T cell epitope described herein. In some embodiments, the pharmaceutical composition comprises a fusion polypeptide described herein. In some embodiments, the pharmaceutical composition comprises a polynucleotide described herein. In some embodiments, the pharmaceutical composition comprises a vector described herein. In some embodiments, the pharmaceutical composition comprises a recombinant virus described herein.

In some embodiments, a pharmaceutical composition disclosed herein comprises an mRNA encoding a polypeptide disclosed herein. Pharmaceutical compositions suitable for in vivo delivery of mRNA to a subject, e.g., a human subject are known to one of skill in the art. See, e.g., US20200261572, US20190351040, and US20190211065, each of which is incorporated herein by reference in its entirety.

In some embodiments, a pharmaceutical composition comprises a polynucleotide encoding a fusion polypeptide comprising: a) immunogenic polypeptide comprising an MHC class II T cell epitope described herein; and b) a SARS-CoV2 spike polypeptide or immunogenic fragment thereof. In some embodiments, the fusion polypeptide comprises an amino acid sequence having at least 70%, at least 80%, at least 85%, at least 90%, at least 95%, at least 97%, at least 98% at least 99% or at least 100% identity with SEQ ID NO:14.

The pharmaceutical compositions described herein are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (22nd ed.), eds. Loyd V. Allen, Jr., 2012, Pharmaceutical Press, Philadelphia, PA, and Encyclopedia of Pharmaceutical Technology, eds. J. Swarbrick and J. C. Boylan, 2013, Marcel Dekker, New York, NY).

### Methods of Use

In one aspect, provided herein are methods of vaccinating a subject comprising administering a therapeutically effective amount of a pharmaceutical composition described herein or an immunogenic composition described herein to the subject. In some embodiments, the immunogenic composition comprises an immunogenic polypeptide comprising an MHC class II T cell epitope described herein. In some embodiments, the immunogenic composition comprises a fusion polypeptide described herein. In some embodiments, the immunogenic composition comprises a polynucleotide described herein. In some embodiments, the immunogenic composition comprises a vector described herein. In some embodiments, the immunogenic composition comprises a recombinant virus described herein. In some embodiments, the pharmaceutical composition comprises an immunogenic polypeptide comprising an MHC class II T cell epitope described herein. In some embodiments, the pharmaceutical composition comprises a fusion polypeptide described herein. In some embodiments, the pharmaceutical composition comprises a polynucleotide described herein. In some embodiments, the pharmaceutical composition comprises a vector described herein. In some embodiments, the pharmaceutical composition comprises a recombinant virus described herein. In some embodiments, the subject is vaccinated against SARS-CoV2 infection. In some embodiments, the immunogenic composition or pharmaceutical composition comprises a polynucleotide encoding a fusion polypeptide comprising: a) immunogenic polypeptide comprising an MHC class II T cell epitope described herein; and b) a SARS-CoV2 spike polypeptide or immunogenic fragment thereof. In some embodiments, the fusion polypeptide comprises an amino acid sequence having at least 70%, at least 80%, at least 85%, at least 90%, at least 95%, at least 97%, at least 98% at least 99% or at least 100% identity with SEQ ID NO:14. In some embodiments, the subject is a human.

In one aspect, provided herein are methods of inducing an immune response in a subject comprising administering an effective amount of a pharmaceutical composition described herein or an immunogenic composition described herein to the subject. In some embodiments, the immunogenic composition comprises an immunogenic polypeptide comprising an MHC class II T cell epitope described herein. In some embodiments, the immunogenic composition comprises a fusion polypeptide described herein. In some embodiments, the immunogenic composition comprises a polynucleotide described herein. In some embodiments, the immunogenic composition comprises a vector described herein. In some embodiments, the immunogenic composition comprises a recombinant virus described herein. In some embodiments, the pharmaceutical composition comprises an immunogenic polypeptide comprising an MHC class II T cell epitope described herein. In some embodiments, the pharmaceutical composition comprises a fusion polypeptide described herein. In some embodiments, the pharmaceutical composition comprises a polynucleotide described herein. In some embodiments, the pharmaceutical composition comprises a vector described herein. In some embodiments, the pharmaceutical composition comprises a recombinant virus described herein. In some embodiments, the immune response is a viral antigen-specific immune response. In some embodiments, the immune response is a SARS-CoV2 specific immune response. In some embodiments, the subject is vaccinated against SARS-CoV2 infection. In some embodiments, the immunogenic composition or pharmaceutical composition comprises a polynucleotide encoding a fusion polypeptide comprising: a) immunogenic polypeptide comprising an MHC class II T cell epitope described herein; and b) a SARS-CoV2 spike polypeptide or immunogenic fragment thereof. In some embodiments, the fusion polypeptide comprises an amino acid sequence having at least 70%, at least 80%, at least 85%, at least 90%, at least 95%, at least 97%, at least 98% at least 99% or at least 100% identity with SEQ ID NO:14. In some embodiments, the immune response is a tumor specific immune response. In some embodiments, the subject is a human. In some embodiments, the subject is a cynomolgus monkey. In some embodiments, the subject is a mouse.

In one aspect, provided herein are methods of treating a viral infection in a subject comprising administering a therapeutically effective amount of a pharmaceutical composition described herein or an immunogenic composition described herein to the subject. In some embodiments, the immunogenic composition comprises an immunogenic polypeptide comprising an MHC class II T cell epitope described herein. In some embodiments, the immunogenic composition comprises a fusion polypeptide described herein. In some embodiments, the immunogenic composition comprises a polynucleotide described herein. In some embodiments, the immunogenic composition comprises a vector described herein. In some embodiments, the immunogenic composition comprises a recombinant virus described herein. In some embodiments, the pharmaceutical composition comprises an immunogenic polypeptide comprising an MHC class II T cell epitope described herein. In some embodiments, the pharmaceutical composition comprises a fusion polypeptide described herein. In some embodiments, the pharmaceutical composition comprises a polynucleotide described herein. In some embodiments, the pharmaceutical composition comprises a vector described herein. In some embodiments, the pharmaceutical composition comprises a recombinant virus described herein. In some embodiments, the viral infection is a SARS-CoV2 infection. In some embodiments, the viral infection is COVID-19. In some embodiments, the subject is vaccinated against SARS-CoV2 infection. In some embodiments, the immunogenic composition or pharmaceutical composition comprises a polynucleotide encoding a fusion polypeptide comprising: a) immunogenic polypeptide comprising an MHC class II T cell epitope described herein; and b) a SARS-CoV2 spike polypeptide or immunogenic fragment thereof. In some embodiments, the fusion polypeptide comprises an amino acid sequence having at least 70%, at least 80%, at least 85%, at least 90%, at least 95%, at least 97%, at least 98% at least 99% or at least 100% identity with SEQ ID NO:14. In some embodiments, the subject is a human.

In one aspect, provided herein are methods of treating a cancer or tumor in a subject comprising administering a therapeutically effective amount of a pharmaceutical composition described herein or an immunogenic composition described herein to the subject. In some embodiments, the immunogenic composition comprises an immunogenic polypeptide comprising an MHC class II T cell epitope described herein. In some embodiments, the immunogenic composition comprises a fusion polypeptide described herein. In some embodiments, the immunogenic composition comprises a polynucleotide described herein. In some embodiments, the immunogenic composition comprises a vector described herein. In some embodiments, the immunogenic composition comprises a recombinant virus described herein. In some embodiments, the pharmaceutical composition comprises an immunogenic polypeptide comprising an MHC class II T cell epitope described herein. In some embodiments, the pharmaceutical composition comprises a fusion polypeptide described herein. In some embodiments, the pharmaceutical composition comprises a polynucleotide described herein. In some embodiments, the pharmaceutical composition comprises a vector described herein. In some embodiments, the pharmaceutical composition comprises a recombinant virus described herein. In some embodiments the pharmaceutical composition or immunogenic composition comprises a neoantigen. In some embodiments, the pharmaceutical composition comprises a recombinant virus described herein. In some embodiments the pharmaceutical composition or immunogenic composition comprises a polynucleotide encoding a neoantigen. In some embodiments, the cancer or tumor is melanoma or glioblastoma. In some embodiments, the cancer or tumor is lung cancer, non-small cell lung cancer, renal cancer, breast cancer, pancreatic cancer, nasopharyngeal cancer, ovarian cancer, cervical cancer, sarcoma, colorectal cancer, HPV16 Associated Cervical Cancer, gastric cancer, or prostate cancer. In some embodiments, the subject is a human.

### EXAMPLES

### Example 1. Identification of Lumazine Synthase CD4 T-Cell Epitopes

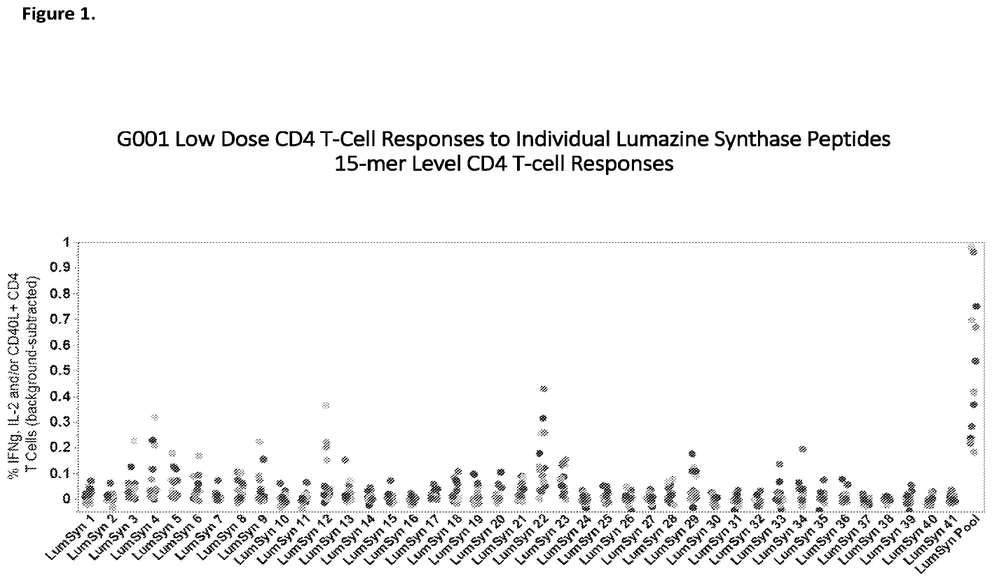

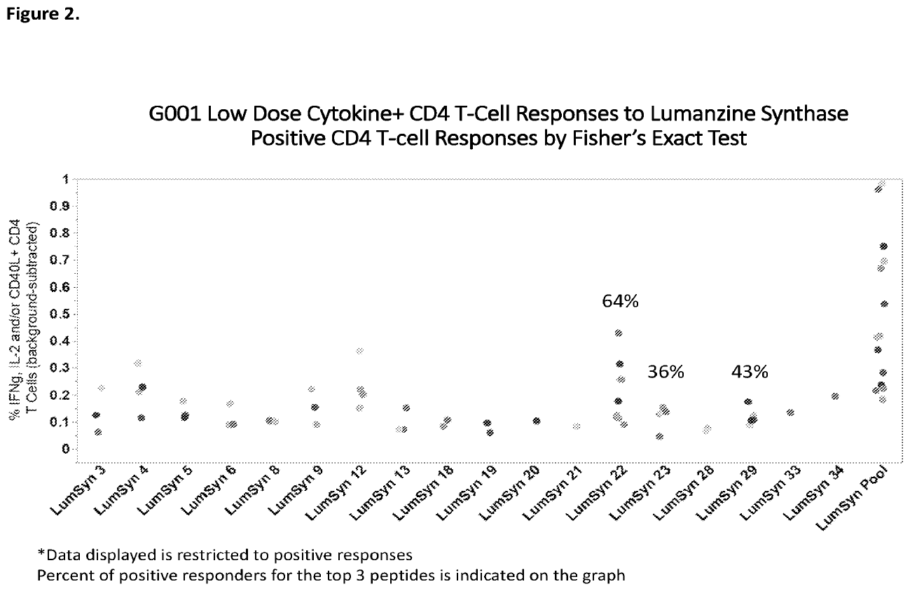

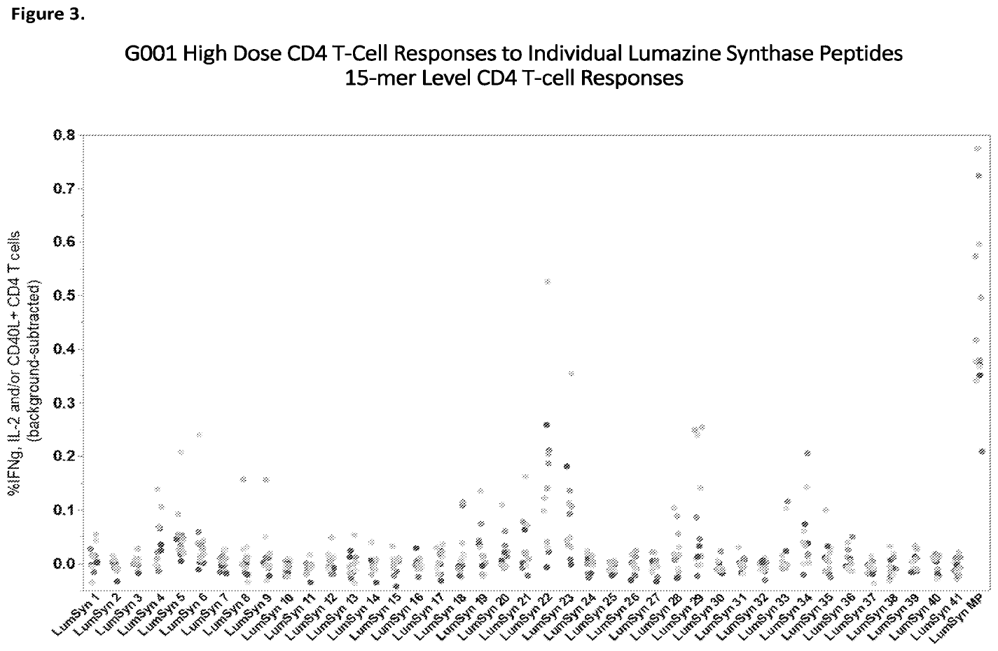

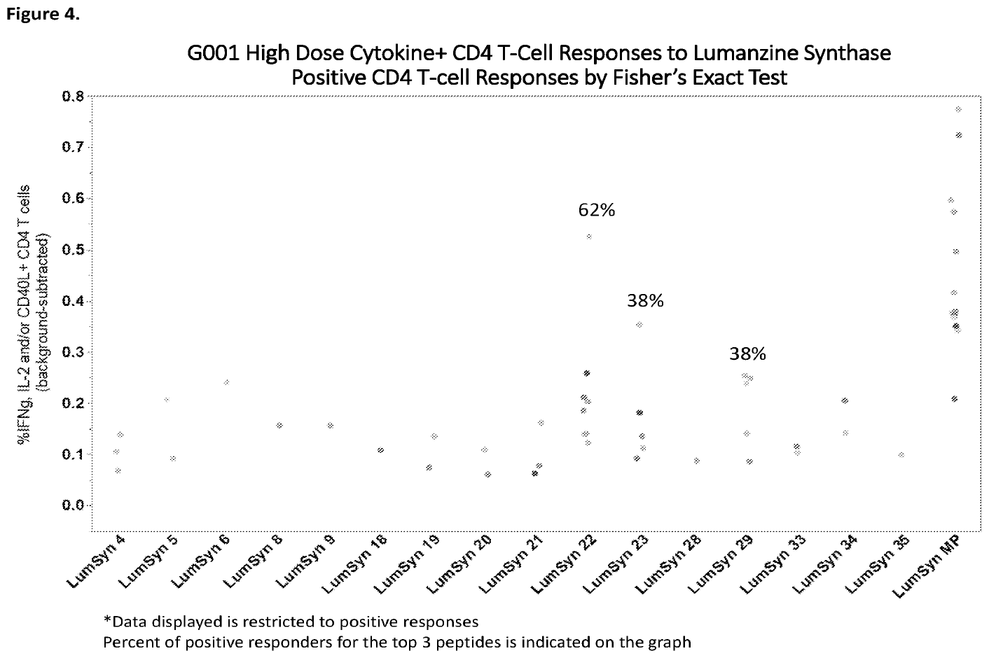

Intracellular cytokine staining (ICS) assay was used to test 41 overlapping 15-mer peptides spanning the full length of A. aeolicus lumazine synthase (SEQ ID NO:5) for their ability to increase INFg, IL-2 and/or CD40L expression in human CD4 T cells. The assay was performed substantially as described in Dintwe 2019, Cytometry A 95(7): 722-725 (2019) using 14 frozen human PBMC samples. The PBMC samples were from 14 participants in a clinical trial in which participants were vaccinated twice with the AS01B-adjuvanted protein eOD-GT8 60mer containing A. aeolicus Lumazine Synthase. In addition to individual 15-mers, a peptide pool comprising all 15-mer peptide was also tested. FIG. 1 shows the results obtained with the panel of peptides. FIG. 2 shows the positive CD-4 T-cell responses by Fisher's Exact Test. Peptides ATPHFDYIASEVSKG (SEQ ID NO:1) (LumSyn22), FGVITADTLEQAIER (SEQ ID NO:2) (LumSyn29) and FDYIASEVSKGLADL (SEQ ID NO:3) (LumSyn23) achieved the highest response rates. 65% (9/14) of vaccine recipients mounted IFNg or IL-2 or CD40L CD4+ T cell responses to the peptide LumSyn22, 36% (5/14) mounted such responses to the peptide LumSyn23, and 43% (6/14) mounted responses to the peptide LumSyn29. Considering combined responses, 71% (10/14) of vaccine recipients responded to LumSyn22 or LumSyn23, 86% (12/14) of vaccine recipients responded to LumSyn22 or LumSyn29, and 93% (13/14) of vaccine recipients responded to LumSyn22 or LumSyn23 or LumSyn29. These results were independently confirmed in a second set of trial participants who received a higher dose of the same vaccine (FIGS. 3 and 4). 62% of high-dose vaccine recipients tested mounted IFNg or IL-2 or CD40L CD4+ T cell responses to the peptide LumSyn22, 38% mounted such responses to the peptide LumSyn23, and 38% mounted responses to the peptide LumSyn29.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

What is claimed is:

1. An immunogenic polypeptide comprising an MHC class II T cell epitope, wherein the peptide comprises an amino acid sequence of a fragment of A. aeolicus lumazine synthase (LS).

2. The immunogenic polypeptide of claim 1, wherein LS comprises an amino acid sequence that has at least 70%, at least 80%, at least 85%, at least 90%, at least 95%, at least 97%, at least 98% at least 99% or at least 100% identity with SEQ ID NO:5.

3. The immunogenic polypeptide of claim 1, wherein LS comprises the amino acid sequence of SEQ ID NO:5.

4. The immunogenic polypeptide of any one of claims 1 to 5, wherein the fragment of LS is a fragment of no more than 60, 55, 50, 45, 40, 35, 30, 25, 20, 19, 18, 17, 16, 15, 14, 13, 12, 11, or 10 amino acids.

5. The immunogenic polypeptide of any one of claims 1 to 5, wherein the fragment of LS is a fragment consisting of about 60, 55, 50, 45, 40, 35, 30, 25, 20, 19, 18, 17, 16, 15, 14, 13, 12, 11, or 10 amino acids.

6. An immunogenic polypeptide comprising one or more MHC class II T cell epitope, wherein the MHC class II T cell epitope comprises an amino acid sequence selected from the group consisting of
a)
17, 16, 15, 14, 13, 12, 11, or 10
consecutive amino acids of
(SEQ ID NO: 6)
LIRGATPHFDYIASEVSKGLADLSLEL;
b)
17, 16, 15, 14, 13, 12, 11, or 10
consecutive amino acids of
(SEQ ID NO: 7)
KPITFGVITADTLEQAIERAGTK;
or
c)
17, 16, 15, 14, 13, 12, 11, or 10
consecutive amino acids of
(SEQ ID NO: 8)
LIRGATPHFDYIASEVSKGLADLSLELRK
PITFGVITADTLEQAIERAGTK

7. An immunogenic polypeptide comprising one or more MHC class II T cell epitope, wherein the MHC class II T cell epitope comprises an amino acid sequence selected from the group consisting of
a)
15 consecutive amino acids of
(SEQ ID NO: 6)
LIRGATPHFDYIASEVSKGLADLSLEL;
b)
15 consecutive amino acids of
(SEQ ID NO: 7)
KPITFGVITADTLEQAIERAGTK;
or
c)
15 consecutive amino acids of
(SEQ ID NO: 8)
LIRGATPHFDYIASEVSKGLADLSLELRK
PITFGVITADTLEQAIERAGTK

8. An immunogenic polypeptide comprising one or more MHC class II T cell epitope, wherein the MHC class II T cell epitope comprises an amino acid sequence selected from the group consisting of
a) 10 consecutive amino acids of
(SEQ ID NO: 6)
LIRGATPHFDYIASEVSKGLADLSLEL;
b) 10 consecutive amino acids of
(SEQ ID NO: 7)
KPITFGVITADTLEQAIERAGTK;
or
c)
10 consecutive amino acids of
(SEQ ID NO: 8)
LIRGATPHFDYIASEVSKGLADLSLE
LRKPITFGVITADTLEQAIERAGTK

9. An immunogenic polypeptide comprising one or more MHC class II T cell epitope, wherein the MHC class II T cell epitope comprises an amino acid sequence selected from the group consisting of
a) 10 consecutive amino acids of
(SEQ ID NO: 1)
ATPHFDYIASEVSKG;
b) 10 consecutive amino acids of
(SEQ ID NO: 2)
FGVITADTLEQAIER;
or
c) 10 consecutive amino acids of
(SEQ ID NO: 3)
FDYIASEVSKGLADL

10. An immunogenic polypeptide comprising one or more MHC class II T cell epitope, wherein the MHC class II T cell epitope comprises an amino acid sequence selected from the group consisting of
a)
(SEQ ID NO: 1)
ATPHFDYIASEVSKG
(LumSyn22) comprising 0, 1, 2, 3,
4, or 5 substitutions,
b)
(SEQ ID NO: 2)
FGVITADTLEQAIER
(LumSyn29) comprising 0, 1, 2, 3,
4, or 5 substitutions,
c)
(SEQ ID NO: 3)
FDYIASEVSKGLADL
(LumSyn23) comprising 0, 1, 2, 3,
4, or 5 substitutions,
d)
(SEQ ID NO: 4)
ATPHFDYIASEVSKGLADL
(LumSyn22/23) comprising 0, 1, 2,
3, 4, or 5 substitutions,
e) 9, 10, 11, 12, 13, 14, or 15
consecutive residues of
(SEQ ID NO: 1)
ATPHFDYIASEVSKG
(LumSyn22),
f) 9, 10, 11, 12, 13, 14, or 15
consecutive residues of
(SEQ ID NO: 2)
FGVITADTLEQAIER
(LumSyn29),
g) 9, 10, 11, 12, 13, 14, or 15
consecutive residues of
(SEQ ID NO: 3)
FDYIASEVSKGLADL
(LumSyn23),
and
h) 10, 11, 12, 13, 14, 15, 16,
17, 18, or 19 consecutive
residues of
(SEQ ID NO: 4)
ATPHFDYIASEVSKGLADL
(LumSyn22/23).

11. The immunogenic polypeptide of claim 10, wherein the MHC class II T cell epitope comprises an amino acid sequence selected from the group consisting of
a) 9, 10, 11, 12, 13, 14, or 15
consecutive residues of
(SEQ ID NO: 1)
ATPHFDYIASEVSKG
(LumSyn22),
b) 9, 10, 11, 12, 13, 14, or 15
consecutive residues of
(SEQ ID NO: 2)
FGVITADTLEQAIER
(LumSyn29),
c) 9, 10, 11, 12, 13, 14, or 15
consecutive residues of
(SEQ ID NO: 3)
FDYIASEVSKGLADL
(LumSyn23),
and
d) 10, 11, 12, 13, 14, 15, 16, 17,
18, or 19 consecutive residues of
(SEQ ID NO: 4)
ATPHFDYIASEVSKGLADL
(LumSyn22/23).

12. The immunogenic polypeptide of claim 10, wherein the MHC class II T cell epitope comprises an amino acid sequence selected from the group consisting of
a)
(SEQ ID NO: 1)
ATPHFDYIASEVSKG,
b)
(SEQ ID NO: 2)
FGVITADTLEQAIER,
c)
(SEQ ID NO: 3)
FDYIASEVSKGLADL,
and
d)
(SEQ ID NO: 4)
ATPHFDYIASEVSKGLADL

13. The immunogenic polypeptide of any one of claims 10 to 12, wherein the immunogenic polypeptide comprises at least 2 MHC class II T cell epitopes.

14. The immunogenic polypeptide of claim 13, wherein the at least 2 MHC class II T cell epitopes comprise the amino acid sequences of
a)
(SEQ ID NO: 1)
ATPHFDYIASEVSKG
(LumSyn22) comprising
0, 1, 2, 3, 4, or 5 substitutions;
and
b)
(SEQ ID NO: 2)
FGVITADTLEQAIER
(LumSyn29) comprising 0, 1, 2, 3, 4,
or 5 substitutions.

15. The immunogenic polypeptide of claim 13, wherein the at least 2 MHC class II T cell epitopes comprise the amino acid sequences of
a)
(SEQ ID NO: 1)
ATPHFDYIASEVSKG
(LumSyn22);
and
b)
(SEQ ID NO: 2)
FGVITADTLEQAIER
(LumSyn29).

16. The immunogenic polypeptide of claim 13, wherein the at least 2 MHC class II T cell epitopes comprise the amino acid sequences of
a)
(SEQ ID NO: 4)
ATPHFDYIASEVSKGLADL
(LumSyn22/23) comprising 0, 1, 2, 3,
4, or 5 substitutions;
and
b)
(SEQ ID NO: 2)
FGVITADTLEQAIER
(LumSyn29) comprising 0, 1, 2, 3, 4,
or 5 substitutions.

17. The immunogenic polypeptide of claim 13, wherein the at least 2 MHC class II T cell epitopes comprise the amino acid sequences of
a)
(SEQ ID NO: 4)
ATPHFDYIASEVSKGLADL
(LumSyn22/23);
and
b)
(SEQ ID NO: 2)
FGVITADTLEQAIER
(LumSyn29).

18. The immunogenic polypeptide of any one of claims 13 to 17, wherein the at least 2 MHC class II T cell epitopes are adjacent to each other in any order.

19. The immunogenic polypeptide of any one of claims 13 to 17, wherein the at least 2 MHC class II T cell epitopes are in any order and are separated by a linker peptide.

20. The immunogenic polypeptide of claim 19, wherein the linker comprises no more than 10 or no more than 5 amino acid residues.

21. The immunogenic polypeptide of claim 19 or claim 20, wherein the linker comprises one or more repeats of the GGS (SEQ ID NO:16) or GGGS (SEQ ID NO:19) sequence.

22. The immunogenic polypeptide of claim 19 or claim 20, wherein the linker comprises the amino acid sequence of GGS (SEQ ID NO:16), GGSGGS (SEQ ID NO:17), GGSGGSGGS (SEQ ID NO:18), GGGS (SEQ ID NO:19), GGGSGGGS (SEQ ID NO:20), or GGGSGGGSGGGS (SEQ ID NO:21).

23. The immunogenic polypeptide of any one of claims 8 to 10, wherein the immunogenic polypeptide comprises the amino acid sequence of ATPHFDYIASEVSKGLADLGGSFGVITADTLEQAIER (SEQ ID NO:12) comprising 0, 1, 2, 3, 4, or 5 substitutions.

24. The immunogenic polypeptide of any one of claims 8 to 12, wherein the immunogenic polypeptide comprises the amino acid sequence having at least 70%, at least 80%, at least 85%, at least 90%, at least 95%, at least 97%, at least 98% at least 99% or at least 100% identity with
(SEQ ID NO: 12)
ATPHFDYIASEVSKGLADLGGSFGVITADTLEQAIER.

25. The immunogenic polypeptide of any one of claims 8 to 12, wherein the immunogenic polypeptide comprises the amino acid sequence of ATPHFDYIASEVSKGLADLGGSFGVITADTLEQAIER (SEQ ID NO:12).

26. The immunogenic polypeptide of any one of claims 8 to 12, wherein the immunogenic polypeptide comprises the amino acid sequence of
(SEQ ID NO: 10)
ATPHFDYIASEVSKGLADLSLELRKPITFGVITADTLEQAIER
comprising 0, 1, 2, 3, 4, or 5 substitutions.

27. The immunogenic polypeptide of any one of claims 8 to 12, wherein the immunogenic polypeptide comprises the amino acid sequence having at least 70%, at least 80%, at least 85%, at least 90%, at least 95%, at least 97%, at least 98% at least 99% or at least 100% identity with
(SEQ ID NO: 10)
ATPHFDYIASEVSKGLADLSLELRKPITFGVITADTLEQAIER.

28. The immunogenic polypeptide of any one of claims 8 to 12, wherein the immunogenic polypeptide comprises the amino acid sequence of
(SEQ ID NO: 10)
ATPHFDYIASEVSKGLADLSLELRKPITFGVITADTLEQAIER.

29. The immunogenic polypeptide of any one of claims 8 to 28, wherein the immunogenic polypeptide consists of between about 10 and 60, between about 10 and 50, between about 10 and 45, between about 10 and 40, between about 10 and 35, between about 10 and 30, between about 10 and 25, between about 10 and 20, or between about 10 and 15 residues.

30. The immunogenic polypeptide of any one of claims 8 to 28, wherein the immunogenic polypeptide is capable of binding to a human MHC class II molecule.

31. A fusion polypeptide comprising at least one immunogenic polypeptide according to any one of claims 1 to 30.

32. A fusion polypeptide comprising
a) at least one immunogenic polypeptide according to any one of claims 1 to 30; and
b) at least one pathogen derived polypeptide or tumor specific neoantigen polypeptide.

33. The fusion polypeptide of claim 32, wherein the at least one pathogen derived polypeptide comprises a viral, bacterial, or parasitic polypeptide.

34. The fusion polypeptide of claim 33, wherein the at least one pathogen derived polypeptide comprises a viral polypeptide.

35. The fusion polypeptide of claim 34, wherein the viral polypeptide comprises a Betacoronavirus, Chikungunya virus, Dengue virus, Ebola virus, Eastern Equine Encephalitis virus, Herpes Simplex virus, Human Cytomegalovirus, Human Papillomavirus. Human Metapneumovirus, Influenza virus, Japanese Encephalitis virus, Marburg virus, Measles, Parainfluenza virus, Respiratory Syncytial virus, Sindbis virus, Varicella Zoster virus. Venezuelan Equine Encephalitis virus, West Nile virus, Yellow Fever virus, or Zika virus polypeptide or an immunogenic fragment thereof.

36. The fusion polypeptide of claim 34, wherein the viral polypeptide comprises a MERS-CoV polypeptide, SARS-CoV polypeptide, SARS-CoV-2 polypeptide or an immunogenic fragment thereof.

37. The fusion polypeptide of claim 34, wherein the viral polypeptide comprises a SARS-CoV polypeptide, SARS-CoV-2 polypeptide or an immunogenic fragment thereof.

38. The fusion polypeptide of claim 34, wherein the viral polypeptide comprises a SARS-CoV-2 spike protein (S), SARS-CoV-2 envelope protein (E), SARS-CoV-2 nucleocapsid protein (N), SARS-CoV-2 membrane protein (M) or an immunogenic fragment thereof.

39. The fusion polypeptide of claim 34, wherein the viral polypeptide comprises a SARS-CoV-2 spike protein (S) or an immunogenic fragment thereof.

40. The fusion polypeptide of any one of claims 32 to 39, wherein the at least one immunogenic polypeptide and the at least one pathogen derived or tumor specific neoantigen polypeptide are directly linked in any order.

41. The fusion polypeptide of any one of claims 32 to 39, wherein the at least one immunogenic polypeptide and the at least one pathogen derived or tumor specific neoantigen polypeptide are separated by a linker.

42. The fusion polypeptide of claim 41, wherein the linker comprises no more than 10 or no more than 5 amino acid residues.

43. The fusion polypeptide of claim 41 or claim 42, wherein the linker comprises one or more repeats of the GGS (SEQ ID NO:16) or GGGS (SEQ ID NO:19) sequence.

44. The fusion polypeptide of claim 41 or claim 42, wherein the linker comprises the amino acid sequence of GGS (SEQ ID NO:16), GGSGGS (SEQ ID NO:17), GGSGGSGGS (SEQ ID NO:18), GGGS (SEQ ID NO:19), GGGSGGGS (SEQ ID NO:20), or GGGSGGGSGGGS (SEQ ID NO:21).

45. An isolated polynucleotide encoding the immunogenic polypeptide of any one of claims 1 to 31 or the fusion polypeptide of any one of claims 32 to 44.

46. The polynucleotide of claim 45 that is DNA.

47. The polynucleotide of claim 45 that is RNA.

48. The polynucleotide of claim 47, wherein the RNA is mRNA comprising modified ribonucleotides.

49. A vector comprising the polynucleotide of claim 45.

50. A recombinant virus comprising the polynucleotide of claim 45.

51. An immunogenic composition comprising the immunogenic polypeptide of any one of claims 1 to 30, the fusion polypeptide of any one of claims 31 to 44, the polynucleotide of any one of claims 45 to 48, the vector of claim 49, or the recombinant virus of claim 50.

52. An immunogenic composition comprising
a) at least one immunogenic polypeptide according to any one of claims 1 to 30; and
b) at least one target antigen.

53. The immunogenic composition of claim 52, wherein the at least one target antigen comprises a hapten.

54. The immunogenic composition of claim 52, wherein the at least one target antigen comprises a pathogen derived antigen or a tumor specific neoantigen.

55. The immunogenic composition of claim 52, wherein the pathogen derived antigen is a viral, bacterial, or parasitic antigen.

56. The immunogenic composition of claim 52, wherein the target antigen comprises a viral, bacterial, or parasitic polypeptide or antigenic fragment thereof or a tumor specific neoantigen.

57. The immunogenic composition of claim 52, wherein the target antigen comprises a viral polypeptide of an antigenic fragment thereof.

58. The immunogenic composition of claim 57, wherein the viral polypeptide is selected from the group consisting of Betacoronavirus, Human Immunodeficiency Virus, Chikungunya virus, Dengue virus, Ebola virus, Eastern Equine Encephalitis virus, Herpes Simplex virus, Human Cytomegalovirus, Human Papillomavirus. Human Metapneumovirus, Influenza virus, Japanese Encephalitis virus, Marburg virus, Measles, Parainfluenza virus, Respiratory Syncytial virus, Sindbis virus, Varicella Zoster virus. Venezuelan Equine Encephalitis virus, West Nile virus, Yellow Fever virus, or Zika virus polypeptide and an immunogenic fragment thereof.

59. The immunogenic composition of claim 57, wherein the viral polypeptide is selected from the group consisting of MERS-CoV polypeptide, SARS-CoV polypeptide, SARS-CoV-2 polypeptide and an immunogenic fragment thereof.

60. The immunogenic composition of claim 57, wherein the viral polypeptide comprises a SARS-CoV-2 polypeptide or an immunogenic fragment thereof.

61. The immunogenic composition of claim 57, wherein the viral polypeptide comprises a SARS-CoV-2 spike protein (S), SARS-CoV-2 envelope protein (E), SARS-CoV-2 nucleocapsid protein (N), SARS-CoV-2 membrane protein (M) or an immunogenic fragment thereof.

62. The immunogenic composition of claim 57, wherein the viral polypeptide comprises a SARS-CoV-2 spike protein (S) or an immunogenic fragment thereof.

63. The immunogenic composition of any one of claims 52 to 62, wherein the immunogenic composition further comprises a carrier, wherein the carrier optionally comprises a liposome, polymeric nanoparticle, hydrogel, micelle, dendrimer, inorganic nanoparticle, virus like particle, protein nanoparticle or combinations thereof.

64. The immunogenic composition of claim 63, wherein the carrier comprises a liposome, polymeric nanoparticle, hydrogel, micelle, dendrimer, inorganic nanoparticle, virus like particle, protein nanoparticle or combinations thereof.

65. The immunogenic composition of any one of claims 51 to 64, further comprising an adjuvant.

66. The immunogenic composition of any one of claims 51 to 64, wherein the immunogenic composition is capable of eliciting an increased immune response in a subject compared to the immune response elicited by a reference immunogenic composition not comprising the polypeptide comprising an MHC class II T cell epitope according to any one of claims 1 to 30.

67. The immunogenic composition of claim 64, wherein the increased immune response is an increased humoral response.

68. The immunogenic composition of claim 64, wherein the increased immune response is an increased cellular immune response.

69. The immunogenic composition of any one of claims 66 to 68, wherein the subject is a mouse or a cynomolgus monkey.

70. A pharmaceutical composition comprising the immunogenic polypeptide of any one of claims 1 to 30, the fusion polypeptide of any one of claims 31 to 44, the polynucleotide of any one of claims 45 to 48, the vector of claim 49, the recombinant virus of claim 50, or the immunogenic composition of any one of claims 51 to 69 and a pharmaceutically acceptable excipient.

71. A method of vaccinating a subject comprising administering a therapeutically effective amount of the immunogenic polypeptide of any one of claims 1 to 30, the fusion polypeptide of any one of claims 31 to 44, the polynucleotide of any one of claims 45 to 48, the vector of claim 49, or the recombinant virus of claim 50, the immunogenic composition of any one of claims 51 to 69, or the pharmaceutical composition of claim 70 to the subject.

72. A method of inducing an immune response in a subject comprising administering an effective amount of the immunogenic polypeptide of any one of claims 1 to 30, the fusion polypeptide of any one of claims 31 to 44, the polynucleotide of any one of claims 45 to 48, the vector of claim 49, or the recombinant virus of claim 50, the immunogenic composition of any one of claims 51 to 69, or the pharmaceutical composition of claim 70 to the subject.

73. The method of claim 72, wherein the immune response is a viral antigen-specific immune response.

74. A method of treating a viral infection in a subject comprising administering a therapeutically effective amount of the immunogenic polypeptide of any one of claims 1 to 30, the fusion polypeptide of any one of claims 31 to 44, the polynucleotide of any one of claims 45 to 48, the vector of claim 49, or the recombinant virus of claim 50, the immunogenic composition of any one of claims 51 to 69, or the pharmaceutical composition of claim 70 to the subject.

75. A method of treating a tumor in a subject comprising administering a therapeutically effective amount of the immunogenic polypeptide of any one of claims 1 to 30, the fusion polypeptide of any one of claims 31 to 44, the polynucleotide of any one of claims 45 to 48, the vector of claim 49, or the recombinant virus of claim 50, the immunogenic composition of any one of claims 51 to 69, or the pharmaceutical composition of claim 70 to the subject.

76. The method of any one of claims 71 to 75, comprising the administration of the fusion polypeptide of any one of claims 31 to 44.

77. The method of any one of claims 71 to 75, comprising the administration of polynucleotide of any one of claims 45 to 48.

78. The method of any one of claims 77, wherein the polynucleotide comprises an mRNA comprising modified ribonucleotides.

79. The method of any one of claims 71 to 78, wherein the subject is a human.

80. A method of producing the immunogenic polypeptide of any one of claims 1 to 30 comprising or the fusion polypeptide of any one of claims 31 to 43 comprising culturing a host cell comprising the polynucleotide of claim 44 under suitable conditions to produce the immunogenic polypeptide or fusion polypeptide.

81. The method of claim 80, wherein the host cell is a CHO cell or a HEK293 cell.


SEQUENCE LISTING (21 sequences)

SEQ ID NO: 1 - protein, 15 aa, Artificial Sequence
  Recombinant polypeptide
ATPHFDYIASEVSKG

SEQ ID NO: 2 - protein, 15 aa, Artificial Sequence
  Recombinant polypeptide
FGVITADTLEQAIER

SEQ ID NO: 3 - protein, 15 aa, Artificial Sequence
  Recombinant polypeptide
FDYIASEVSKGLADL

SEQ ID NO: 4 - protein, 19 aa, Artificial Sequence
  Recombinant polypeptide
ATPHFDYIASEVSKGLADL

SEQ ID NO: 5 - protein, 154 aa, Artificial Sequence
  Recombinant polypeptide
MQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 6 - protein, 27 aa, Artificial Sequence
  Recombinant polypeptide
LIRGATPHFDYIASEVSKGLADLSLEL

SEQ ID NO: 7 - protein, 23 aa, Artificial Sequence
  Recombinant polypeptide
KPITFGVITADTLEQAIERAGTK

SEQ ID NO: 8 - protein, 51 aa, Artificial Sequence
  Recombinant polypeptide
LIRGATPHFDYIASEVSKGLADLSLELRKPITFGVITADTLEQAIERAGTK

SEQ ID NO: 9 - protein, 39 aa, Artificial Sequence
  Recombinant polypeptide
ATPHFDYIASEVSKGLADLSLGGSFGVITADTLEQAIER

SEQ ID NO: 10 - protein, 43 aa, Artificial Sequence
  Recombinant polypeptide
ATPHFDYIASEVSKGLADLSLELRKPITFGVITADTLEQAIER

SEQ ID NO: 11 - protein, 1261 aa, Artificial Sequence
  Recombinant polypeptide
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 37 aa, Artificial Sequence
  Recombinant polypeptide
ATPHFDYIASEVSKGLADLGGSFGVITADTLEQAIER

SEQ ID NO: 13 - protein, 195 aa, Artificial Sequence [sequence omitted]

SEQ ID NO: 14 - protein, 274 aa, Artificial Sequence
  Recombinant polypeptide
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGSGGSGGSATPHFDYIASEVSKGLADLSLGGSFDYIASEVSKGLADLGGSGGSGGSKSSIASFFFIIGLIIGLFLVLR

SEQ ID NO: 15 - protein, 22 aa, Artificial Sequence
  Recombinant polypeptide
KSSIASFFFIIGLIIGLFLVLR

SEQ ID NO: 16 - protein, 3 aa, Artificial Sequence
  Recombinant polypeptide
GGS

SEQ ID NO: 17 - protein, 6 aa, Artificial Sequence
  Recombinant polypeptide
GGSGGS

SEQ ID NO: 18 - protein, 9 aa, Artificial Sequence
  Recombinant polypeptide
GGSGGSGGS

SEQ ID NO: 19 - protein, 4 aa, Artificial Sequence
  Recombinant polypeptide
GGGS

SEQ ID NO: 20 - protein, 8 aa, Artificial Sequence
  Recombinant polypeptide
GGGSGGGS

SEQ ID NO: 21 - protein, 12 aa, Artificial Sequence
  Recombinant polypeptide
GGGSGGGSGGGS